(19) United States
(12) Patent Application Publication
(10) Pub. No.: US 2026/0001950 A1
(43) Pub. Date: Jan. 1, 2026

(54) METHODS OF ENHANCING EFFICACY OF IMMUNOCONJUGATES USING PAIRED HUMAN ANTIBODIES TARGETED AT NON-OVERLAPPING B-CELL EPITOPES ON THE SAME ANTIGEN

(71) Applicant: Medicovestor, Inc., Wilmington, DE (US)

(72) Inventor: Seah Lim, Wilmington, DE (US)

(21) Appl. No.: 19/055,378

(22) Filed: Feb. 17, 2025

Related U.S. Application Data
(60) Provisional application No. 63/666,523, filed on Jul. 1, 2024.
(60) Provisional application No. 63/666,595, filed on Jul. 1, 2024.

(51) Int. Cl. C07K 16/28; A61K 39/00; A61K 47/68; A61K 51/10

(52) U.S. Cl. CPC C07K 16/28; A61K 47/6849; A61K 51/1045; A61K 2039/507; C07K 2317/21; C07K 2317/92

(57) ABSTRACT

This disclosure describes methods and drug therapies for treating or preventing various health conditions, including cancer. The methods and therapies of the present disclosure comprise antibody-based therapies that functionally increase antibody binding to target antigens. Specifically, the antibody-based therapies increase the copy number of an antigen by utilizing at least a pair of differing antibodies directed at non-overlapping epitopes of the same antigen for effectively delivering payloads to a target cell. Each differing antibody may be conjugated to the same or a differing payload. The antibody-based therapy may use proteins that specifically bind human folate receptor alpha (FRalpha) with high affinity. These proteins include a human anti-FRalpha IgG1 that is suitable for use as a therapeutic antibody to treat conditions, such as cancers that express FRalpha. Other FRalpha-binding proteins are also described including antibody fragments, antibody conjugates, and fusion proteins.

### CROSS REFERENCE TO RELATED APPLICATIONS

This application claims the benefit of U.S. Provisional Patent Application No. 63/666,523, filed Jul. 1, 2024, and U.S. Provisional Patent Application No. 63/666,595, filed Jul. 1, 2024, each of which is incorporated by reference herein in its entirety.

### SEQUENCE LISTING

This disclosure includes a sequence listing, which has file name “1200590038_Sequence_Listing.xml,” which was created on Feb. 17, 2025, which has a file size of 54000 bytes, and which is incorporated by reference in its entirety.

### FIELD OF THE DISCLOSURE

The invention relates to antibody drug conjugates (ADCs) and more specifically to methods of enhancing the efficacy of immunoconjugate compounds by functionally increasing the copy number of a targeted antigen by utilizing a combination of antibodies directed at distinct and non-overlapping B-cell epitopes for the treatment or prevention of various health conditions, including cancer.

### BACKGROUND

Folate receptor alpha (FRalpha) is a glycosyl-phosphatidylinositol (GPI)-linked cell-surface glycoprotein receptor for folates. It shows a restricted normal tissue expression, typically being expressed at low levels in the choroid plexus, lungs, and kidneys. It is, however, expressed in high levels in serous and endometrioid epithelial ovarian cancer, endometrial adenocarcinoma, and non-small cell lung cancer of the adenocarcinoma subtype. FRalpha expression is maintained in metastatic foci and recurrent carcinomas in ovarian cancer patients and has also been observed to be maintained after chemotherapy in patients treated for epithelial ovarian and endometrial cancers. FRalpha is therefore considered a tumor-associated antigen.

A number of different monoclonal antibodies have been developed to treat cancer by targeting FRalpha. Certain monoclonal antibodies (MoAbs) have demonstrated efficacy as powerful protein molecules capable of mediating tumor cell cytolysis. Naked MoAbs do so by way of either inducing complement-mediated cell lysis or antibody-dependent cellular cytotoxicity (ADCC). In some instances, ADCC is the predominant mode of tumor cell killing. It has been found that the binding specificity of MoAbs to their target can be leveraged for delivering either chemotherapy agents such as, in some instances, antibody-drug conjugates (ADCs) or in others, radioisotopes (including radioimmunoconjugates (RICs)) to the site of the tumor cells so that exposure to the chemotherapy or radiation is more specific and intense, reducing non-specific toxicity and damage to healthy tissues. In some cases, this also increases the number of tumor cells killed. That is, therapies that utilize ADCs, typically rely on the antibody as a carrier to deliver a payload (e.g., chemotherapy agent, cytotoxic drugs, radioisotopes) to the tumor cells, where the payload is responsible for killing the tumor cells.

The abundance of the antigen is indicated by the antigen copy number which provides the number of copies of an antigen on a cell surface. The efficacy of MoAb-based therapies is dependent on how abundant the expression of the antigen is on the tumor cells (e.g., the antigens copy number).

As proteins, MoAbs or portions of the MoAbs, such as their variable regions, may themselves elicit an immune response in the host cell. This is particularly seen in murine-derived MoAbs. As a result, it has been observed that patients treated with mouse-derived MoAbs frequently develop a human anti-mouse antibody (HAMA) response that may preclude further therapy for the patient, thus hampering the practical use of such therapies.

FRalpha has been the target of mirvetuximab soravtansine (elahere) which is an ADC currently approved by the FDA for treating platinum-resistant ovarian cancer. However, to date it has only been approved for patients whose tumor cells express more than 70% of FRalpha, thereby narrowing the potential market for this ADC.

### SUMMARY

Various aspects of this disclosure relate to the discovery of a novel antibody-based therapy that targets an antigen of a tumor cell via antibody-drug conjugates (ADCs). The novel therapy, subject of the present disclosure, increases the number of antibodies and/or ADC molecules that are able to attach to a target tumor cell with a fixed number of antigens by functionally increasing the antigen copy number. A typical ADC molecule is designed to include a single antibody, such as immunoglobulin G (IgG), and a single type of payload. The efficacy of these typical ADC molecules relies on the antigen copy number which correlates to the number of antibody molecules that are able to bind to a tumor cell surface. This binding may be accomplished either by targeting tumors that express a high density of a targetable antigen or by the use of other strategies that increase the number of antibodies binding to the tumor cells without an actual increase in the number of antigens present on the surface of the tumor cells. Therapies of the present disclosure advantageously increase the functional copy number of the target antigen by attaching antibodies to non-overlapping epitopes of a target antigen.

Embodiments of the present disclosure relate to methods of treating and preventing various health conditions, including cancer, by employing more than one immunoconjugate for targeting a cancer-related antigen, such as FRalpha. Each immunoconjugate attaches to a non-overlapping epitope of the same antigen and may carry the same payload, or different payloads. Advantageously, the antibody-based therapies of the present disclosure may deliver at least twice the number of payloads to a target cell as compared to conventional monoclonal antibody-based ADC therapies. Additionally, as the antibodies can be conjugated with different payloads, the payloads may be synergistically designed to treat a patient with a combination of therapies to improve efficacy and/or reduce side effects of the treatment. Therapies of the present disclosure enhance the intrinsic antitumor properties of the backbone antibodies (i.e., the naked antibody, without a payload) by Antibody-Dependent Cell-Mediated Cytotoxicity (ADCC), Antibody-Dependent Cellular Phagocytosis (ADCP), and Complement-dependent cytotoxicity (CDC).

In one non-limiting example, a therapy of the present disclosure may employ two different antibodies, such as IgG antibodies, where one of the antibodies binds to a first epitope of the target antigen (e.g., FRalpha) and the other binds to a second epitope of the target antigen, where the first epitope and the second epitope do not overlap. The two antibodies may by conjugated with the same payload. Alternatively, one of the antibodies may be conjugated with a first payload and the other antibody may be conjugated with a second payload.

Various aspects of the disclosure relate to methods and therapeutic agents to functionally increase the copy number of a target antigen of a human subject. The method may comprise administering, to the human subject, a set of immunotherapeutic agents that target an antigen, wherein the set of immunotherapeutic agents may comprise: a first immunotherapeutic agent comprising a first antibody conjugated with a first payload; a least one second immunotherapeutic agent comprising a second antibody conjugated with a second payload, wherein the first antibody and the second antibody are different and bind different, non-overlapping epitopes of the antigen, and wherein the first payload and the second payload are the same or different.

In some examples, the set of immunotherapeutic agents consist of the first immunotherapeutic agent and a second immunotherapeutic agent, the first immunotherapeutic agent targeting a first epitope of the antigen and the second immunotherapeutic agent targeting a second epitope of the antigen, wherein the first epitope and the second epitope are distinct and non-overlapping.

In some examples, the first payload, the second payload, or both comprises a radioactive isotope selected from the group comprising actinium-225, astatine-211, bismuth-212, bismuth-213, copper-67, gallium-68, holmium-166, iodine-124, iodine-131, lutetium-177, samarium-153, technetium-99, terbium-149, and yttrium-90.

In some examples, the first payload, the second payload, or both comprises a pharmaceutical agent comprising a moiety selected from the group comprising amanitin, 3-aminophenyl hemiasterlin, calicheamicin, camptothecin, deruxtecan, doxorubicin, emtansine, eribulin, exatecan, irinotecan, maleimidocaproyl monomethyl auristatin F, maytansine, mertansine (N2′-deacetyl-N2′-(3-mercapto-1-oxopropyl)-maytansine; DM1), monomethyl auristatin F, paclitaxel, PE38, pyrrolobenzodiazepine, ravtansine (N2′-deacetyl-N2′-(4-mercapto-4-methyl-1-oxopentyl) maytansine; DM4), SN-38, and vedotin.

In some examples, the antigen is folate receptor alpha.

In some examples, the set of immunotherapeutic agents are administered to target an antigen and kill a cell associated with the target antigen via antibody-dependent cell-mediated cytotoxicity (ADCC), antibody-dependent cellular phagocytosis (ADCP), or complement-dependent cytotoxicity (CDC).

In some examples, the set of immunotherapeutic agents are administered via in an antibody-drug conjugates (ADC) or radioimmunoconjugates (RIC) formulation.

In some examples, the epitopes are b-cell epitopes.

In some examples, the first payload and the second payload are the same.

In some examples, the first payload and the second payload are different.

In some examples, the immunotherapeutic agent is an IgG antibody.

In some examples, the immunotherapeutic agent is administered at an effective amount that is effective to induce cell death in at least a portion of the cells that express folate receptor alpha.

In some examples, the method and therapeutic agents modulate the cells that express folate receptor alpha by inducing cell death.

Various aspects of the disclosure relate to methods and therapeutic agents of treating or preventing cancer in a human subject. In some examples, the human subject presents with cancer; and at least a portion of the cells that express folate receptor alpha are cancer cells.

In some examples, the method may comprise identifying that the human subject comprises cells that overexpress folate receptor alpha.

In some examples, the cells that overexpress folate receptor alpha comprise epithelial cells, fallopian tube cells, leukemia cells, epithelial cells, or a combination thereof.

In some examples, the method may comprise identifying that a tissue sample of the human subject comprises either RNA encoding folate receptor alpha or folate receptor alpha protein prior to the administering.

In some examples, the tissue sample is a blood sample.

In some examples, the tissue sample is a biopsy.

In some examples, the administering is selected from intravenous, intramuscular, subcutaneous, intradermal, intraocular, parenteral, intraperitoneal, intrathecal, intralesional, and intratumoral administering.

Various aspects of the disclosure relate a set of immunotherapeutic agents for functionally increasing the copy number of a target antigen of a human subject. The set of immunotherapeutic agents may comprise a first immunotherapeutic agent comprising a first antibody conjugated with a first payload; a least one second immunotherapeutic agent comprising a second antibody conjugated with a second payload, wherein the first antibody and the second antibody are different and bind different, non-overlapping epitopes of the antigen, and wherein the first payload and the second payload are the same or different.

The preceding Background and Summary sections are provided as a brief introduction to the described subject matter as well as a synopsis of some of the technological improvements and advantages that it provides. The Background and Summary shall not be construed as identifying essential aspects of the described subject matter, nor shall they be construed to limit the interpretation of this specification or any patent claim that matures from this specification.

### BRIEF DESCRIPTION OF THE DRAWINGS

A further understanding of this specification may be appreciated with reference to the following drawings. The drawings are exemplary, and neither this specification nor any patent claim that matures from this specification shall be construed as limited by the drawings.

FIG. 1A is a diagram of an exemplary pair of immunoconjugates, each immunoconjugate comprising a payload (payload 1 and payload 2) conjugated to an IgG antibody, where each immunoconjugate targets a differing, non-overlapping epitope (E1 and E2) of a cancer-related antigen, such as FRalpha, according to embodiments of the present disclosure.

FIG. 1B is a computer model of an exemplary pair of immunoconjugates, each immunoconjugate comprising a payload (payload 1 and payload 2) conjugated to an IgG antibody (IgG1), where each immunoconjugate targets a differing, non-overlapping epitope (E1 and E2) of a cancer-related antigen, such as FRalpha, according to embodiments of the present disclosure.

FIGS. 2A and 2B are graphs that depict the binding of various anti-FRalpha IgG antibodies to human FRalpha extracellular domain as assessed by enzyme-linked immunosorbent assay (ELISA). The dashed line depicts binding of the control anti-FRalpha IgG mirvetuximab.

FIGS. 3A and 3B are graphs that depict the binding of various anti-FRalpha IgG antibodies to monkey FRalpha extracellular domain as assessed by enzyme-linked immunosorbent assay (ELISA). The dashed line depicts binding of the control anti-FRalpha IgG mirvetuximab.

FIGS. 4A and 4B are graphs that depict the competition effect of various anti-FRalpha IgG antibodies to human FRalpha extracellular domain as assessed by enzyme-linked immunosorbent assay (ELISA). The dashed line depicts binding of the control anti-FRalpha IgG mirvetuximab.

FIGS. 5A and 5B are graphs that depict the binding of various anti-FRalpha IgG antibodies to FRalpha expressed on HEK293 cells that have been engineered to over-express the human FRalpha, assessed by flow cytometry. The dashed line depicts binding of the control anti-FRalpha IgG mirvetuximab.

FIG. 6 is a graph that depicts the binding of various anti-FRalpha IgG antibodies to HEK293 cells that have been engineered to over-express the human FRalpha, assessed by flow cytometry.

FIG. 7A is a diagram that depicts the mechanics of biolayer inferometric (BLI) principle for determining inhibition rate of a saturating antibody (Ab1) and a competing antibody (Ab2) in binding to a capture antigen (Ag), such as FRalpha.

FIG. 7B is a graph that depicts the mechanics of biolayer inferometric (BLI) principle for determining inhibition rate of a saturating antibody (Ab1) and a competing antibody (Ab2) in binding to a capture antigen (Ag), such as FRalpha.

FIG. 8 is a graph that depicts the inhibition rate of various anti-FRalpha IgG antibodies, disclosed herein, in binding to a capture antigen, FRalpha, determined via inferometric (BLI) principle.

FIGS. 9A through 9E are graphs that depict affinity measurements of various anti-FRalpha IgG antibodies at various concentrations, disclosed herein, to FRalpha measured by signal recognition particle (SRP).

FIG. 10 is a computer model based on the amino acid sequences disclosed herein, illustrating one embodiment of the invention in which one FRalpha antibodies are directed at two non-overlapping B-cell epitopes on the FRalpha, comprising an extracellular domain, a transmembrane domain, and an intracellular domain.

### DETAILED DESCRIPTION

Various aspects of this disclosure relate to methods and therapies for enhancing the efficacy of immunoconjugate compounds by utilizing multiple non-linked antibodies directed at distinct and non-overlapping epitopes of a single target antigen to functionally increase the copy number of the target antigen. Functionally increasing the copy number of a targeted antigen increases the number of antibodies that are able to bind to problematic cells (e.g., tumor cells) carrying the target antigen, and thus increase the quantity of payloads delivered to the problematic cells.

Immunoconjugates of the present disclosure may be presented in an antibody-drug conjugate (ADC) and in a radioimmunoconjugates (RIC) formulations. The methods and therapies disclosed herein comprise administering multiple monoclonal antibodies each conjugated to cytotoxic payloads via a chemical linker that are directed toward a target antigen expressed on a target cells surface. The cytotoxic payloads are configured to be released from the antibodies upon attachment or ingestion into the target cell, thereby reducing systemic exposure and toxicity. Each immunoconjugate may carry a distinct payload, or the immunoconjugates may carry the same payload. In the case of distinct payloads, the probability of the therapy being successful and overall efficacy of the therapy may be improved as the (chemotherapy) exposure is increased. In the case of attaching the same payload to each antibody, efficacy of the treatment may be improved as the amount of payload delivered to each antigen is increased, even if the antigen copy number of a patient is low as the (chemotherapy) exposure is increased.

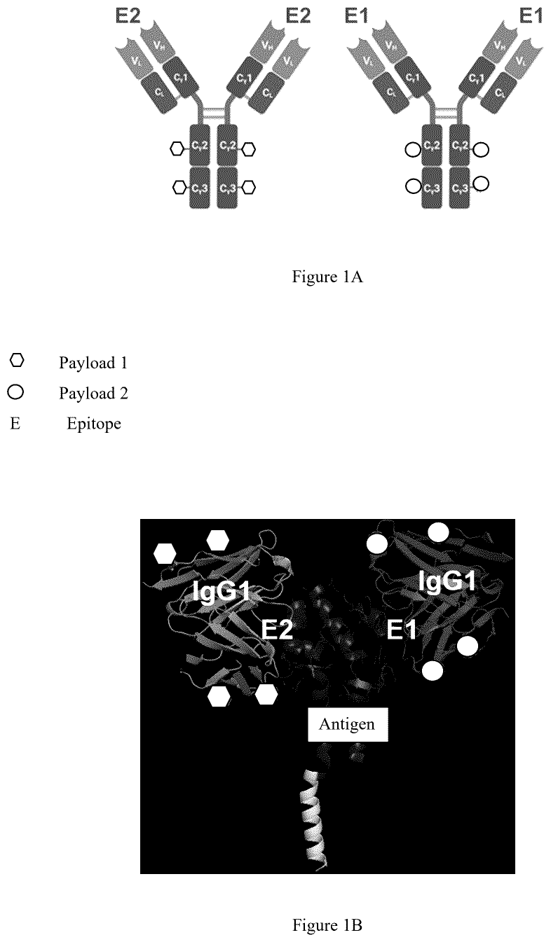

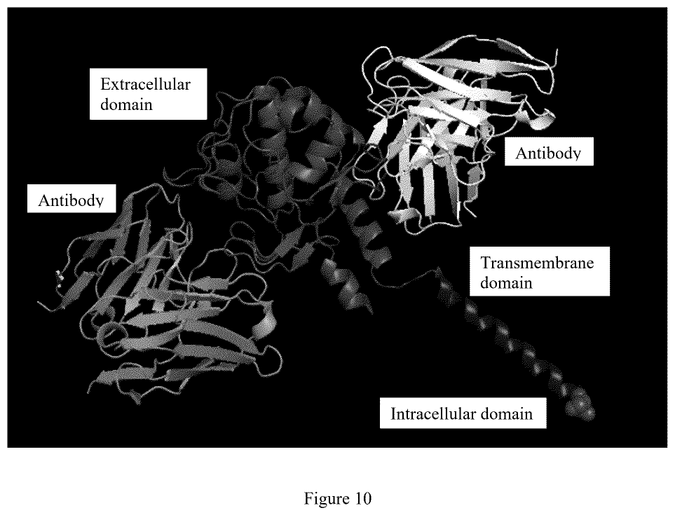

In embodiments of the present disclosure, methods and therapies comprise administering at least a pair of antibody immunoconjugates to a patient, where each antibody binds to a distinct, non-overlapping epitope of the same antigen. Embodiments of the present disclosure are directed to a formulation of ADCs that comprises or consists of two antibodies targeting two non-overlapping epitopes of an antigen, as depicted in FIGS. 1A, 1B, and 10. In a non-limiting example, each antibody of the pair is conjugated with the same cytotoxic payload (e.g., payload 1 and payload 2 are the same payload). In another non-limiting example, a first antibody of the pair is conjugated with a first payload (e.g., payload 1), and the second antibody of the pair is conjugated with a second payload (e.g., payload 2), where the first and second payloads are different. That is, a formulation for combination treatment (e.g., chemotherapy) ADCs may be generated that comprises or consists of two antibodies, each carrying a synergistic payload, targeting two non-overlapping epitopes of a target antigen.

In embodiment, antibodies of the present disclosure target folate receptor alpha (FRa, FRalpha). The epitopes may be configured to target non-overlapping B-cell epitopes on FRalpha.

Embodiments of the present disclosure utilize a pair of FRalpha v-genes or/and their gene product in therapeutic development of, and not limited to, naked antibodies, bispecific antibodies, BiTEs, ADC, RIC, and cell-based therapy such as chimeric antigen receptor (CAR)-T, CAR-NK, and CAR-monocytes.

Therapies of the present disclosure enhance the intrinsic antitumor properties of the backbone antibodies (i.e., the naked antibody, without a payload) by Antibody-Dependent Cell-Mediated Cytotoxicity (ADCC), Antibody-Dependent Cellular Phagocytosis (ADCP), and Complement-dependent cytotoxicity (CDC).

Embodiments of the present disclosure are directed to antibodies that display high target specificity and affinity for FRalpha, described in further detail below with reference to SEQ ID NO: 1-44, below. According to embodiments of the present disclosure, methods and treatments comprise administering a combination of immunoconjugates comprising an IgG antibody and a payload to a patient for the treatment or prevention of various health conditions, including cancer. The combination of immunoconjugates comprises at least two different antibodies that each target and bind to distinct and non-overlapping epitopes of the same antigen, such as FRalpha. The number of different antibodies used in the treatment therapy may be based on the target antigen, as the number of epitopes typically depends on the antigen's size. In embodiments, the combination of immunoconjugates consists of two different antibodies. In embodiments, the combination of immunoconjugates comprises more than two different antibodies. The combination of immunoconjugates may comprise a single payload. That is, the same payload may be conjugated to each of the differing antibodies. The combination of immunoconjugates may comprise more than one payload. In some cases, each differing antibody may be conjugated with a different payload. That is, for example, a first antibody may be conjugated with a first payload, and a second antibody may be conjugated with a second payload, where the first and second antibodies differ and the first and second payloads differ. In some cases, the same antibodies may be conjugated with the differing payloads. That is, a first set of a first antibody may be conjugated with a first payload and a second set of the first antibody may be conjugated with a second payload, where the first and second payload are different. Similarly, a first set of a second antibody may be conjugated with a third payload and a second set of the second antibody may be conjugated with a fourth payload. The first antibody and the second antibody may be different. Each of the first, second, third, and fourth payloads may be different. Alternatively, some of the first, second, third, and fourth payloads may be the same. For example, the first and third payload may be the same. It should be expressly understood that these combinations are merely examples, and the present disclosure is directed to methods and therapies comprising a combination of any number of distinct immunoconjugates that each bind to a non-overlapping epitope of a target antigen, where each distinct immunoconjugate comprises an antibody and a payload.

By way of treatment of a health condition that involves targeting an antigen, the combination of immunoconjugates may be administered at the same time. Alternatively, the treatment may include step wise administration, where a portion of the combination of immunoconjugates is administered first and a second portion (or remaining portion) of the combination of immunoconjugates is administered sometime later. Administration may be based on administering the same payload at the same or differing times, administering the same antibody at the same or differing times, or a combination thereof. In some embodiments, the combination of immunoconjugates is packaged in a single formulation (e.g., single vial of ADC) for administration to a patient. In other embodiments, the immunoconjugates may be packaged in multiple formulations (e.g., separate vials of ADC). Each formulation may comprise a single ADC or multiple ADCs. In a non-limiting example, a first vial comprises a first set of one or more antibodies and a second vial comprises a second set of one or more antibodies. In another embodiment, each immunoconjugate may be packaged in separate formulation (e.g., separate vials of ADC). In a non-limiting example, a first vial comprises first antibody, a second vial comprises a second antibody, and so on such that each antibody is packaged in a distinct vial. The multiple formulations may be administered to a patient at the same time, such that the patient receives antibodies simultaneously. Alternatively, the patient may receive one or more of the formulations at different times. The antibodies of the combination of immunoconjugates, whether formulated in a single vial or multiple vials, may be formulated in any ratio, but particularly in equal parts. For example, the combination of immunoconjugates comprises two antibodies that are formulated in a 1:1 ratio, which may be allocated to separate vials of ADC or in a single vial of ADC.

Various aspects of this disclosure relate to an anti-FRalpha antibody or antigen-binding fragment thereof, which comprises sequence homology (or sequence identity) with the full chain and/or VH and VL variable regions disclosed herein. In all embodiments, the antibody or antigen-binding fragment thereof binds human FRalpha.

The term “antibody” includes immunoglobulins (Ig's) of different classes (for example, IgA, IgG, IgM, IgD, and IgE) and subclasses (for example, IgG1, IgG2a, and IgG4) and also includes mouse antibodies, fully-human antibodies, chimeric human/animal antibodies, and engineered variants thereof. This specification describes, for example, a fully-human IgG1 antibody.

The term “antigen-binding fragment” refers to both Fab fragments and single-chain variable fragments (ScFv). scFvs are fusion proteins of two variable regions connected with a flexible linker, which fusion proteins retain antigen-binding properties comparable to a Fab. Examples of scFvs include brolucizumab (also known as BEOVU®).

As used in this disclosure, the term “sequence homology” refers to percent “positives” as determined by Standard Protein BLAST® over the full length of a sequence set forth in a SEQ ID NO. Standard Protein BLAST® is available at https://blast.ncbi.nlm.nih.gov/Blast.cgi?PROGRAM=blastp. BLAST® is generally described in Altschul, et al. (1997), “Gapped BLAST and PSI-BLAST: a new generation of protein database search programs”, Nucleic Acids Res. 25:3389-3402, and in Altschul, et al. (2005) “Protein database searches using compositionally adjusted substitution matrices”, FEBS J. 272:5101-5109. As used in this disclosure, the term “sequence identity” refers to the percent of exact matches over the full length of a sequence set forth in a SEQ ID NO.

In all embodiments, the anti-FRalpha antibody or antigen-binding fragment thereof comprises two variable domains. The two variable domains include a heavy chain variable region and a light chain variable region.

Mutations to the VH and/or VL amino acid sequences herein may be engineered, for example, to tune the avidity of an antibody (or antigen-binding fragment thereof) to the FRalpha antigen, to modulate expression or glycosylation, and/or for other purposes.

In some embodiments, the anti-FRalpha antibody or antigen-binding fragment thereof has a KD with human FRalpha of no greater than 50 nanomolar. In some specific embodiments, the anti-FRalpha antibody or antigen-binding fragment thereof has a KD with human FRalpha of no greater than 25 nanomolar. In some very specific embodiments, the anti-FRalpha antibody or antigen-binding fragment thereof has a KD with human FRalpha of no greater than 10 nanomolar.

In some embodiments, KD is determined by ELISA.

In some embodiments, the anti-FRalpha antibody comprises an IgG heavy chain constant region. In some specific embodiments, the anti-FRalpha antibody comprises an IgG1 heavy chain constant region. In some specific embodiments, the anti-FRalpha antibody comprises an IgG4 heavy chain constant region.

In some embodiments, the t anti-FRalpha antibody comprises a kappa or lambda light chain constant region. In some specific embodiments, the anti-FRalpha antibody comprises a kappa light chain constant region.

In some embodiments, the anti-FRalpha antibody comprises a human heavy chain constant region and a human light chain constant region. In some specific embodiments, the anti-FRalpha antibody comprises a human IgG heavy chain constant region and a human kappa or lambda light chain constant region. In some very specific embodiments, the anti-FRalpha antibody comprises a human IgG1 heavy chain constant region and a human kappa light chain constant region.

In some embodiments, a primary anti-FRalpha antibody disclosed herein comprises a first and second variable domain paired in the primary antibody such that the first and second variable domains bind an epitope of human FRalpha. A secondary anti-FRalpha antibody disclosed herein comprises a third variable domain and a fourth variable domain that are paired in the secondary antibody such that the third and fourth variable domains bind a different epitope. It should be expressly understood that primary and secondary as used herein to describe antibodies does not imply an order of administration to a patient. That is, the primary and secondary antibodies disclosed herein may be administered to a patient in any order, or simultaneously.

In this disclosure, the term “joined” refers a spatial proximity and orientation between VH and VL regions that allow the VH and VL regions to simultaneously bind an epitope. VH and VL regions may be joined, for example, in a Fab by quaternary structure that includes one or more disulfide bonds and non-covalent interactions between the heavy chain constant domain CH1 and the light chain constant domain CL. VH and VL regions may also be joined, for example, as a scFv fusion protein, which generally includes a flexible linker, such as polyglycine, that tethers the VH and VL regions in spatial proximity and permits spatial orientations in which the VH and VL regions can simultaneously bind an epitope.

Various aspects of this disclosure relate to an antibody conjugate comprising a anti-FRalpha antibody as described anywhere in this disclosure, wherein the anti-FRalpha antibody is conjugated to a payload, such as a radioactive isotope or a pharmaceutical agent.

In some embodiments, the primary and/or secondary anti-FRalpha antibody is conjugated to a radioactive isotope. In some specific embodiments, the primary and/or secondary anti-FRalpha antibody is conjugated to a radioactive isotope selected from actinium-225, astatine-211, bismuth-212, bismuth-213, copper-67, gallium-68, holmium-166, iodine-124, iodine-131, lutetium-177, samarium-153, technetium-99, terbium-149, and yttrium-90. Examples of antibodies conjugated to radioactive isotopes include tositumomab (also known as BEXXAR®) and ibritumomab tiuxetan (also known as ZEVALIN®). Those of ordinary skill are capable of designing antibodies that are conjugated to radioactive isotopes using known strategies such as those used to conjugate iodine-131 in tositumomab and to conjugate yttrium-90 or indium-111 to ibritumomab (see, for example, U.S. Pat. Nos. 6,565,827 and 7,422,739, which are incorporated by reference in their entirety).

In some embodiments, the primary and/or secondary anti-FRalpha antibody is conjugated to a pharmaceutical agent. In some specific embodiments, the primary and/or secondary anti-FRalpha antibody is conjugated to a moiety selected from amanitin, 3-aminophenyl hemiasterlin, calicheamicin, camptothecin, deruxtecan, doxorubicin, emtansine, eribulin, exatecan, irinotecan, maleimidocaproyl monomethyl auristatin F, maytansine, mertansine (N2′-deacetyl-N2′-(3-mercapto-1-oxopropyl)-maytansine; DM1), monomethyl auristatin F, paclitaxel, PE38, pyrrolobenzodiazepine, ravtansine (N2′-deacetyl-N2′-(4-mercapto-4-methyl-1-oxopentyl) maytansine; DM4), SN-38, and vedotin. Examples of antibodies conjugated to pharmaceutical agents include gemtuzumab ozogamicin (also known as MYLOTARG®) and trastuzumab emtansine (also known as KADCYLA®). Those of ordinary skill are capable of designing antibodies that are conjugated to pharmaceutical agents using known strategies such as those used to conjugate calicheamicin to gemtuzumab and emtansine to trastuzumab (see, for example, U.S. Pat. Nos. 5,877,296 and 8,088,387, which are incorporated by reference in their entirety). Examples of anti-FRalpha immunoconjugates include farletuzumab ecteribulin, luveltamab tazevibulin, and mirvetuximab soravtansine (also known as ELAHERE®), which are described, for example, in U.S. Pat. Nos. 10,322,192, 10,596,270, 10,752,683, which are incorporated by reference in their entirety.

Various aspects of this disclosure relate to a pharmaceutical composition comprising a primary and at least one secondary anti-FRalpha antibody, antigen-binding fragments thereof, or conjugates thereof as described anywhere in this disclosure and a pharmaceutically acceptable carrier. Suitable pharmaceutically acceptable carriers generally include water with dissolved solutes that buffer pH and provide metal cations and an ionic strength that stabilize an antibody or other therapeutic of this disclosure. Such formulations are generally sterile, and the selection and preparation of such pharmaceutically acceptable carriers are well known. Solid formats including lyophilized therapeutics generally include, for example, metal cations, anions, and optionally polyols such as sugars (for example, trehalose or glucose) that stabilize the therapeutic in the solid phase and during its reconstitution into an aqueous format. General guidance on selecting pharmaceutically acceptable carriers is available, for example, in REMINGTON: THE SCIENCE AND PRACTICE OF PHARMACY, 22nd edition (Allen Jr, Loyd V., editor) Pharmaceutical Press, 2012, and the skilled practitioner will also look to the formulations of the therapeutics described in this disclosure as well as other existing therapeutics in selecting a pharmaceutically acceptable carrier. Such guidance is generally available in the scientific literature and on existing product labels.

In some embodiments, the pharmaceutical composition is suitable for administration to a subject. In some specific embodiments, the pharmaceutical composition is suitable for administration to a human patient. In some very specific embodiments, the pharmaceutical composition is suitable for intravenous administration to a human patient.

In some embodiments, the pharmaceutical composition comprises a plurality (i.e., more than one) of anti-FRalpha antibodies, and each antibody has a purity of at least 85 percent relative to total protein in the pharmaceutical composition. In some specific embodiments, the pharmaceutical composition comprises a plurality of FRalpha antibodies, and each antibody has a purity of at least 90 percent relative to total protein in the pharmaceutical composition. In some very specific embodiments, the pharmaceutical composition comprises a plurality of anti-FRalpha antibodies, and each antibody has a purity of at least 95 percent relative to total protein in the pharmaceutical composition.

In some embodiments, purity is determined by chromatography. In some specific embodiments, purity is determined by high-performance liquid chromatography (HPLC).

Various aspects of this disclosure relate to a kit, comprising (1) a hermetically-sealed container that contains a pharmaceutical composition as described anywhere in this specification and (2) instructions for use of the pharmaceutical composition.

Various aspects of this disclosure relate to a medical device, comprising the pharmaceutical composition as described anywhere in this specification. In some embodiments, the medical device is a syringe, a venous cannula, or a drug-eluting implant.

Various aspects of this disclosure relate to a method of treating or preventing various health condition, such as cancer, in a subject, comprising identifying that the subject comprises cells that express or overexpress a target antigen, such as FRalpha, and administering a pharmaceutical composition as described anywhere in this disclosure. In one example, determining that the subject comprises cells that express FRalpha include, for example, identifying mRNA that encodes FRalpha by real-time reverse transcription-polymerase chain reaction (RT-PCR) and identifying FRalpha protein expression by flow cytometry and/or immunohistochemistry. Such methods may advantageously allow determination that a cell (e.g., a cancer cell) expresses FRalpha, for example, based upon the prior selection of one or more cells (e.g., cancer cells) for analysis.

Various aspects of this disclosure relate to a method of treating or preventing various medical condition, such as cancer, in a subject, comprising identifying that the subject comprises cells that express a target antigen, such as FRalpha, above a certain threshold and administering a pharmaceutical composition as described anywhere in this disclosure. In one example, determining that the subject comprises cells that express FRalpha above a certain threshold include, for example, identifying mRNA that encodes FRalpha by RT-PCR and identifying FRalpha protein expression by flow cytometry and/or immunohistochemistry. Such methods may advantageously allow determination that a cell (e.g., a cancer cell) expresses FRalpha above a certain threshold, for example, based upon the prior selection of one or more cells (e.g., a cancer cell) for analysis.

Various aspects of this disclosure relate to a method to modulate cells that express FRalpha in a human subject, comprising administering the immunotherapeutic agent to the human subject, wherein the immunotherapeutic agent is or comprises multiple anti-FRalpha antibodies or antigen-binding fragments thereof as described anywhere in this disclosure. In some embodiments, the method is a method of treating or preventing cancer in a human subject. In some specific embodiments, the method is a method of treating or preventing cancer in a human subject, the human subject presents with cancer, and at least a portion of the cells that express FRalpha are cancer cells.

In some embodiments the immunotherapeutic agent is administered at an effective amount that is effective to induce cell death in at least a portion of the cells that express FRalpha, and the method modulates the cells that express FRalpha by inducing cell death. In some specific embodiments, the immunotherapeutic agents comprise an Fc region, and the method induces cell death by Fc receptor mediated binding and activation of one or more leukocytes to the cells that express FRalpha. In some specific embodiments, at least a portion of the immunotherapeutic agents are conjugated to a radioactive isotope, and the method induces cell death by emitting radiation within or in proximity to the cells that express FRalpha. In some specific embodiments, at least a portion of the immunotherapeutic agents are conjugated to a pharmaceutical agent, the pharmaceutical agent is cytotoxic, and the method induces cell death by releasing the pharmaceutical agent within or in proximity to the cells that express FRalpha. In some very specific embodiments, the immunotherapeutic agents are conjugated to a pharmaceutical agent by a labile linker, the pharmaceutical agent is cytotoxic, and the method induces cell death by releasing the pharmaceutical agent within or in proximity to the cells that express FRalpha.

In some embodiments, the subject is a mammal. In some specific embodiments, the subject is a rodent, lagomorph, feline, canine, porcine, ovine, caprine, lama, bovine, equine, or primate. In some very specific embodiments, the subject is a human patient.

In some embodiments, the subject is male or female. In some specific embodiments, the subject is female. In some specific embodiments, the subject is male.

In some embodiments, the subject presents with elevated expression of FRalpha. In some specific embodiments, the subject presents with cancer, and cells of the cancer express an elevated concentration of FRalpha.

In some embodiments, the method comprises identifying that the subject comprises cells that express FRalpha.

In some embodiments, the subject presents with cancer. In some specific embodiments, the subject presents with cancer, and the cancer comprises cells that express FRalpha. In some very specific embodiments, the subject presents with cancer, and the cancer comprises cells that express an aberrantly high concentration of FRalpha. The cancer cells, for example, may overexpress FRalpha.

In some embodiments, the cancer is selected from ovarian cancer, fallopian tube cancer, primary peritoneal cancer, endometrial cancer, breast cancer, lung cancer, adenocarcinoma, mesothelioma, colorectal cancer, brain cancer, and acute myeloid leukemia. In some specific embodiments, the cancer is non-small cell lung cancer, adenocarcinoma, mesothelioma, triple-negative breast cancer, epithelial ovarian cancer, fallopian tube cancer, primary peritoneal cancer, or acute myeloid leukemia.

In some embodiments, the administering is selected from intravenous, intramuscular, subcutaneous, intradermal, intraocular, parenteral, intraperitoneal, intrathecal, intralesional, and intratumoral administration. In some specific embodiments, the administering is intravenous administration.

Various aspects of this disclosure relate to nucleic acids encoding anti-FRalpha antibodies or antigen-binding fragments thereof as described anywhere in this disclosure.

In some embodiments, the protein is an anti-FRalpha antibody as described anywhere in this disclosure.

In some embodiments, the protein is an antigen-binding fragment of an anti-FRalpha antibody as described anywhere in this disclosure.

In some embodiments, the protein is a bi-specific T-cell engager (BiTE). A BITE is a fusion protein comprising a scFV that binds an antigen (for example, FRalpha) and a domain that binds a cell-surface protein expressed by a T-cell such as CD3. Examples of BiTEs include blinatumomab (also known as BLINCYTO®) and tebentafusp (also known as KIMMTRAK®). Those of ordinary skill are capable of designing BiTEs that bind FRalpha and CD3, for example, by replacing the variable regions that bind CD19 in blinatumomab or glycoprotein 100 in tebentafusp with the variable regions disclosed herein (see, for example, U.S. Pat. Nos. 10,517,960 and 11,597,766, which are incorporated by reference in their entirety).

In some embodiments, the protein is a chimeric antigen receptor (CAR). A CAR is a fusion protein comprising (1) an N-terminal, extracellular scFV that binds an antigen (for example, FRalpha), (2) a transmembrane alpha-helix, and (3) a C-terminal, intracellular signaling domain such as CD3-zeta and/or one or more intracellular signaling domains selected from CD27, CD28, CD134, and CD137. The intracellular signaling domain typically comprises an immunoreceptor tyrosine-based activation motif (ITAM). Examples of CARs include tisagenlecleucel (also known as KYMRIAH®) and axicabtagene ciloleucel (also known as YESCARTA®). Those of ordinary skill are capable of designing CARs that bind FRalpha, for example, by replacing the variable regions that bind CD19 in tisagenlecleucel or axicabtagene ciloleucel with the variable regions disclosed herein (see, for example, U.S. Pat. Nos. 7,446,190, 7,741,465, and 9,499,629, which are incorporated by reference in their entirety).

In some embodiments, the nucleic acid comprises an origin of replication, wherein the nucleic acid is a plasmid.

Various aspects of this disclosure relate to a viral vector, comprising at least one of nucleic acid as described anywhere in this disclosure. In a non-limiting example, a first viral vector may comprise a nucleic acid of a first antibody and a second viral vector may comprise a nucleic acid of a second antibody for a pair of antibodies that target non-overlapping epitopes of the same antigen. In some embodiments, one or both of the first and second viral vectors have a tropism for human leukocytes. In some specific embodiments, one or both of the first and second viral vectors have a tropism for human T-cells, natural killer cells, or monocytes. In some very specific embodiments, one or both of the viral vectors are an adenovirus vector, an adeno-associated virus vector, a lentiviral vector, or a gamma-retroviral vector. Viral vectors may be used, for example, to introduce a nucleic acid encoding a CAR into a leukocyte to produce a transgenic leukocyte that expresses the CAR for use as a cancer immunotherapy.

Various aspects of this disclosure relate to a cell, comprising a nucleic acid as described anywhere in this disclosure or a viral vector as described anywhere in this disclosure.

In some embodiments, the cell is a prokaryote such as E. coli, and the cell is used for cloning or propagating the nucleic acids of the present disclosure. When the cell is a prokaryote, then the nucleic acids generally include an origin of replication for propagation as well as an antibiotic resistance gene to provide a selective advantage for cells that comprise the nucleic acids.

In some embodiments, the cell is a bacterial cell.

In some embodiments, the cell is mammalian cell. In some specific embodiments, the cell is an immortalized mammalian cell line. In some very specific embodiments, the cell is a CHO cell (Chinese hamster ovary), a HEK cell (human embryonic kidney), such as a HEK293 cell, a NS0 cell (murine myeloma cell), a Sp2/0 cell (murine myeloma cell), or a PER.C6® cell (human retina cell). Immortalized mammalian cell lines are generally used to express proteins for use as therapeutics such as therapeutic antibodies (including, for example, the human anti-FRalpha antibody described herein). Mammalian cells generally ensure the fidelity of the tertiary and quaternary structure of the variable regions and other regions of an FRalpha-binding protein as well as the fidelity of post-translational modifications such as glycosylation patterns. Mammalian cells may also be used, for example, to manufacture viral vectors as described herein. The nucleic acid may be present in the cell either transiently or stably.

In some embodiments, the cell is a mammalian cell, and the mammalian cell expresses the protein.

In some embodiments, the cell is a mammalian cell, the mammalian cell expresses a viral vector, and the nucleic acid comprises a packaging signal for packaging the nucleic acid in the viral vector.

In some embodiments, the cell is a tissue culture cell. In some specific embodiments, the cell is a tissue culture cell, and the tissue culture cell expresses the protein. In some specific embodiments, the cell is a tissue culture cell; the tissue culture cell expresses a viral vector; and the nucleic acid comprises a packaging signal for packaging the nucleic acid in the viral vector.

In some embodiments, the cell is a peripheral blood mononuclear cell (PBMC). In some specific embodiments the cell is a human PBMC. In some specific embodiments, the cell is a T-cell, a natural killer cell, a monocyte, a macrophage, or a dendritic cell. In some very specific embodiments, the cell is a human T-cell, a human natural killer cell, a human monocyte, a human macrophage, or a human dendritic cell. PBMCs may be transfected with a nucleic acid to express a CAR such that the PBMCs may be used as a cancer immunotherapy, for example, such as a CAR-T cell, a CAR-NK cell, CAR-monocyte, CAR-macrophage, or CAR-DC.

In some embodiments, the cell is a leukocyte, and the nucleotide sequence encodes a protein that is a CAR. In some specific embodiments, the cell is a human leukocyte, and the nucleotide sequence encodes a protein that is a CAR. In some very specific embodiments, the cell is a human leukocyte, the nucleotide sequence encodes a protein that is a CAR, and the human leukocyte expresses the CAR.

In some embodiments, the cell is a PBMC, and the nucleotide sequence encodes a protein that is a CAR. In some specific embodiments, the cell is a human PBMC, and the nucleotide sequence encodes a protein that is a CAR. In some very specific embodiments, the cell is a human PBMC, the nucleotide sequence encodes a protein that is a CAR, and the human PBMC expresses the CAR.

In some embodiments, the cell is a T-cell, a natural killer cell, a monocyte, a macrophage, or a dendritic cell, and the nucleotide sequence encodes a protein that is a CAR. In some specific embodiments, the cell is a human T-cell, a human natural killer cell, a human monocyte, a human macrophage, or a human dendritic cell, and the nucleotide sequence encodes a protein that is a CAR. In some very specific embodiments, the cell is a human T-cell, a human natural killer cell, a human monocyte, a human macrophage, or a human dendritic cell, the nucleotide sequence encodes a protein that is a CAR, and the cell expresses the CAR.

Various aspects of this disclosure relate to a Fab fragment that binds FRalpha and that comprises variable domains that have sequence homology with the amino acid sequences set forth in SEQ ID NO: 3, 4, 7, 8, 11, 12, 15, 16, and 38-44. A Fab may comprise amino acid sequences that have, for example, at least 90, 95, 97, 98, or 99 percent sequence homology with the sequences set forth in SEQ ID NO: 3, 4, 7, 8, 11, 12, 15, 16, and 38-44 or at least 90, 95, 97, 98, or 99 percent sequence identity or homology with the sequences set forth in SEQ ID NO: 7, 8, 11, 12, 15, 16, and 38-44. In some embodiments, the Fab fragment is encoded by a nucleic acid as described anywhere in this disclosure.

Various aspects of this disclosure relate to an antibody comprising a Fab fragment described anywhere in this disclosure. In some embodiments, the antibody is encoded by a nucleic acid as described anywhere in this disclosure.

Embodiments of the present disclosure are directed to antibodies that display high target specificity and affinity for FRalpha. A primary antibody of the present disclosure comprises an immunoglobulin heavy chain variable region (VH) with the nucleotide sequence of SEQ ID NO: 1 and an immunoglobulin light chain variable region (VL) with the nucleotide sequence of SEQ ID NO: 2, which nucleotide sequences are provided in Table 1, below. The VH amino acid sequence is of SEQ ID NO: 3, and the VL amino acid sequence is of SEQ ID NO: 4. The VH and VL amino acid sequences are provided in Table 2, below.

A secondary antibody of the present disclosure comprises an immunoglobulin VH with the nucleotide sequence of SEQ ID NO: 5 and an immunoglobulin VL with the nucleotide sequence of SEQ ID NO: 6, which nucleotide sequences are provided in Table 3, below. The VH amino acid sequence is of SEQ ID NO: 7, and the VL amino acid sequence is of SEQ ID NO: 8. The VH and VL amino acid sequences are provided in Table 4, below.

A secondary antibody of the present disclosure comprises an immunoglobulin VH with the nucleotide sequence of SEQ ID NO: 9 and an immunoglobulin VL with the nucleotide sequence of SEQ ID NO: 10, which nucleotide sequences are provided in Table 5, below. The VH amino acid sequence is of SEQ ID NO: 11, and the VL amino acid sequence is of SEQ ID NO: 12. The VH and VL amino acid sequences are provided in Table 6, below.

A secondary antibody of the present disclosure comprises an immunoglobulin VH with the nucleotide sequence of SEQ ID NO: 13 and an immunoglobulin VL with the nucleotide sequence of SEQ ID NO: 14, which nucleotide sequences are provided in Table 7, below. The VH amino acid sequence is of SEQ ID NO: 15, and the VL amino acid sequence is of SEQ ID NO: 16. The VH and VL amino acid sequences are provided in Table 8, below.

Various aspects of this disclosure relate to a nucleic acid, comprising a nucleotide sequence that encodes a protein that comprises an antigen-binding region, wherein (1) the antigen-binding region comprises a first variable domain and a second variable domain; and (2) the first variable domain and the second variable domain are paired in the antigen-binding region of the protein such that the antigen-binding region specifically binds human FRalpha.

In some embodiments, the first variable domain comprises a nucleotide sequence that has at least 90 percent sequence identity or homology (i.e., at least 95, 97, 98, 99, 99.5, 99.9 percent sequence identity or homology) with SEQ ID NO: 1. In some specific embodiments, the first variable domain comprises a nucleotide sequence that has at least 95 percent sequence identity or homology (i.e., at least 97, 98, 99, 99.5, 99.9 percent sequence identity or homology) with SEQ ID NO: 1. In some even more specific embodiments, the first variable domain comprises a nucleotide sequence that has at least 99 percent sequence identity or homology (i.e., at least 99.5, 99.9 percent sequence identity or homology) with SEQ ID NO: 1. In some very specific embodiments, the first variable domain comprises a nucleotide sequence that is identical to SEQ ID NO: 1.

In some embodiments, the first variable domain comprises an amino acid sequence that has at least 90 percent sequence identity or homology with SEQ ID NO: 3. In some specific embodiments, the first variable domain comprises an amino acid sequence that has at least 95 percent sequence identity or homology with SEQ ID NO: 3. In some even more specific embodiments, the first variable domain comprises an amino acid sequence that has at least 99 percent sequence identity or homology with SEQ ID NO: 3. In some very specific embodiments, the first variable domain comprises an amino acid sequence that is identical to SEQ ID NO: 3.

In some embodiments, the second variable domain comprises an amino acid sequence that has at least 90 percent sequence identity or homology with SEQ ID NO: 2. In some specific embodiments, the second variable domain comprises an amino acid sequence that has at least 95 percent sequence identity or homology with SEQ ID NO: 2. In some even more specific embodiments, the second variable domain comprises an amino acid sequence that has at least 99 percent sequence identity or homology with SEQ ID NO: 2. In some very specific embodiments, the second variable domain comprises an amino acid sequence that is identical to SEQ ID NO: 2.

In some embodiments, the second variable domain comprises an amino acid sequence that has at least 90 percent sequence identity or homology with SEQ ID NO: 4. In some specific embodiments, the second variable domain comprises an amino acid sequence that has at least 95 percent sequence identity or homology with SEQ ID NO: 4. In some even more specific embodiments, the second variable domain comprises an amino acid sequence that has at least 99 percent sequence identity or homology with SEQ ID NO: 4. In some very specific embodiments, the second variable domain comprises an amino acid sequence that is identical to SEQ ID NO: 4.

In some embodiments, the first variable domain comprises a nucleotide sequence that has at least 90 percent sequence identity or homology with SEQ ID NO: 1, and the second variable domain comprises a nucleotide sequence that has at least 90 percent sequence identity or homology with SEQ ID NO: 2. In some specific embodiments, the first variable domain comprises a nucleotide sequence that has at least 95 percent sequence identity or homology with SEQ ID NO: 1, and the second variable domain comprises a nucleotide sequence that has at least 95 percent sequence identity or homology with SEQ ID NO: 2. In some even more specific embodiments, the first variable domain comprises a nucleotide sequence that has at least 99 percent sequence identity or homology with SEQ ID NO: 1, and the second variable domain comprises a nucleotide sequence that has at least 99 percent sequence identity or homology with SEQ ID NO: 2. In some very specific embodiments, the first variable domain comprises a nucleotide sequence that is identical to SEQ ID NO: 1, and the second variable domain comprises a nucleotide sequence that is identical to SEQ ID NO: 2.

In some embodiments, the first variable domain comprises an amino acid sequence that has at least 90 percent sequence identity or homology with SEQ ID NO: 3, and the second variable domain comprises an amino acid sequence that has at least 90 percent sequence identity or homology with SEQ ID NO: 4. In some specific embodiments, the first variable domain comprises an amino acid sequence that has at least 95 percent sequence identity or homology with SEQ ID NO: 3, and the second variable domain comprises an amino acid sequence that has at least 95 percent sequence identity or homology with SEQ ID NO: 4. In some even more specific embodiments, the first variable domain comprises an amino acid sequence that has at least 99 percent sequence identity or homology with SEQ ID NO: 3, and the second variable domain comprises an amino acid sequence that has at least 99 percent sequence identity or homology with SEQ ID NO: 4. In some very specific embodiments, the first variable domain comprises an amino acid sequence that is identical to SEQ ID NO: 3, and the second variable domain comprises an amino acid sequence that is identical to SEQ ID NO: 4.

In some embodiments, the first variable domain comprises a nucleotide sequence that has at least 90 percent sequence identity or homology with SEQ ID NO: 5, 9, or 13. In some specific embodiments, the first variable domain comprises a nucleotide sequence that has at least 95 percent sequence identity or homology with SEQ ID NO: 5, 9, or 13. In some even more specific embodiments, the first variable domain comprises a nucleotide sequence that has at least 99 percent sequence identity or homology with SEQ ID NO: 5, 9, or 13. In some very specific embodiments, the first variable domain comprises a nucleotide sequence that is identical to SEQ ID NO: 5, 9, or 13.

In some embodiments, the first variable domain comprises an amino acid sequence that has at least 90 percent sequence identity or homology with SEQ ID NO: 7, 11, or 15. In some specific embodiments, the first variable domain comprises an amino acid sequence that has at least 95 percent sequence identity or homology with SEQ ID NO: 7, 11, or 15. In some even more specific embodiments, the first variable domain comprises an amino acid sequence that has at least 99 percent sequence identity or homology with SEQ ID NO: 7, 11, or 15. In some very specific embodiments, the first variable domain comprises an amino acid sequence that is identical to SEQ ID NO: 7, 11, or 15.

In some embodiments, the second variable domain comprises an amino acid sequence that has at least 90 percent sequence identity or homology with SEQ ID NO: 6, 10, or 14. In some specific embodiments, the second variable domain comprises an amino acid sequence that has at least 95 percent sequence identity or homology with SEQ ID NO: 6, 10, or 14. In some even more specific embodiments, the second variable domain comprises an amino acid sequence that has at least 99 percent sequence identity or homology with SEQ ID NO: 6, 10, or 14. In some very specific embodiments, the second variable domain comprises an amino acid sequence that is identical to SEQ ID NO: 6, 10, or 14.

In some embodiments, the second variable domain comprises an amino acid sequence that has at least 90 percent sequence identity or homology with SEQ ID NO: 8, 12, or 16. In some specific embodiments, the second variable domain comprises an amino acid sequence that has at least 95 percent sequence identity or homology with SEQ ID NO: 8, 12, or 16. In some even more specific embodiments, the second variable domain comprises an amino acid sequence that has at least 99 percent sequence identity or homology with SEQ ID NO: 8, 12, or 16. In some very specific embodiments, the second variable domain comprises an amino acid sequence that is identical to SEQ ID NO: 8, 12, or 16.

In some embodiments, the first variable domain comprises a nucleotide sequence that has at least 90 percent sequence identity or homology with SEQ ID NO: 5, 9, or 13, and the second variable domain comprises a nucleotide sequence that has at least 90 percent sequence identity or homology with SEQ ID NO: 6, 10, or 14. In some specific embodiments, the first variable domain comprises a nucleotide sequence that has at least 95 percent sequence identity or homology with SEQ ID NO: 5, 9, or 13, and the second variable domain comprises a nucleotide sequence that has at least 95 percent sequence identity or homology with SEQ ID NO: 6, 10, or 14. In some even more specific embodiments, the first variable domain comprises a nucleotide sequence that has at least 99 percent sequence identity or homology with SEQ ID NO: 5, 9, or 13, and the second variable domain comprises a nucleotide sequence that has at least 99 percent sequence identity or homology with SEQ ID NO: 6, 10, or 14. In some very specific embodiments, the first variable domain comprises a nucleotide sequence that is identical to SEQ ID NO: 5, 9, or 13, and the second variable domain comprises a nucleotide sequence that is identical to SEQ ID NO: 6, 10, or 14.

In some embodiments, the first variable domain comprises an amino acid sequence that has at least 90 percent sequence identity or homology with SEQ ID NO: 7, 11, or 15, and the second variable domain comprises an amino acid sequence that has at least 90 percent sequence identity or homology with SEQ ID NO: 8, 12, or 16. In some specific embodiments, the first variable domain comprises an amino acid sequence that has at least 95 percent sequence identity or homology with SEQ ID NO: 7, 11, or 15, and the second variable domain comprises an amino acid sequence that has at least 95 percent sequence identity or homology with SEQ ID NO: 8, 12, or 16. In some even more specific embodiments, the first variable domain comprises an amino acid sequence that has at least 99 percent sequence identity or homology with SEQ ID NO: 7, 11, or 15, and the second variable domain comprises an amino acid sequence that has at least 99 percent sequence identity or homology with SEQ ID NO: 8, 12, or 16. In some very specific embodiments, the first variable domain comprises an amino acid sequence that is identical to SEQ ID NO: 7, 11, or 15, and the second variable domain comprises an amino acid sequence that is identical to SEQ ID NO: 8, 12, or 16.

The affinity of the antibodies associated with SEQ ID NO: 1-16 for FRalpha was determined to be sub-micromolar by ELISA and flow cytometry and the competitions effects between the antibodies associated with SEQ ID NO: 1-16 were determined by ELISA, as described in more detail with reference to Examples 1-5.

The nucleotide sequences of the human VH and VL regions of the primary antibody are set forth in Table 1 below. The variable regions have the amino acid sequences set forth in Table 2 below. In the context of the IgG1, the primary antibody has a molecular weight of about 143.5 kilodaltons.

TABLE 1
Nucleotide sequences of the variable domains
of the primary anti-FRalpha antibody

| SEQ | | Sequence |
|---|---|---|
| ID | | 10 20 30 40 |
| NO. | Region | 12345678901234567890123456789012345678901234567890 |
| 1 | VH | GAGGTGCAGCTGTTGGAGTCTGGGGGAGGCTTGGTCAAGCCTGGGGGGTC |
| | | CCTGAGACTCTCCTGTGCAGCCTCTGGATTCACCTTCAGTAGCTATACCA |
| | | TGAACTGGGTCCGCCAGGCTCCAGGGAAGGGGCTGGAATGGGTCTCATCC |
| | | ATTAGTAGTGGTGGTGGTTACATATACTACGCAGACTCAGTGAAGGACCG |
| | | ATTCACCATCTCCAGAGACAACGCCAAGAACTCACTGTATCTGCAAATGA |
| | | ACAGCCTGCGAGCCGAGGACACGGCCGTGTATTACTGTGCAAAAGGGGAT |
| | | ACGATCATGATAATATCGGCGTGGGGCCAAGGGACAACGGTCACCGTCTC |
| | | ATCA |
| 2 | VL | GACATCCAGTTGACCCAGTCTCCTTCCACCCTGTCTGCATCTGTAGGAGA |
| | | CAGAGTCACCATCACTTGCCGGGCCAGTCAGAGTATTAGTAGTTGGTTGG |
| | | CCTGGCATCAGCAGAAACCAGGGAAAGCCCCTAAACTCCTCATCTATGCT |
| | | GCATCCAGTTTGCAAAATGGGGTCCCATCAAGGTTCAGCGGCAGTGGATT |
| | | TGGGACAGATTTCACTCTCACCATCAGCAGCCTGCAGCCTGAAGACTTTG |
| | | CAACTTACTATTGTCAACAGGCTAGCAGTTTCCCTCTCACTTTCGGCGGA |
| | | GGGACCAAGGTGGAGATCAAA |

TABLE 2
Amino acid sequences of the variable
domains of the primary anti-FRalpha antibody

| SEQ | | Sequence |
|---|---|---|
| ID | | 10 20 30 40 |
| NO. | Region | 12345678901234567890123456789012345678901234567890 |
| 3 | VH | EVQLLESGGGLVKPGGSLRLSCAASGFTFSSYTMNWVRQAPGKGLEWVSS |
| | | ISSGGGYIYYADSVKDRFTISRDNAKNSLYLQMNSLRAEDTAVYYCAKGD |
| | | TIMIISAWGQGTTVTVSS |
| 4 | VL | DIQLTQSPSTLSASVGDRVTITCRASQSISSWLAWHQQKPGKAPKLLIYA |
| | | ASSLQNGVPSRFSGSGFGTDFTLTISSLQPEDFATYYCQQASSFPLTFGG |
| | | GTKVEIK |

The nucleotide sequences of the human VH and VL regions of a secondary antibody, according to embodiments of the present disclosure, are set forth in Table 3 below. The variable regions have the amino acid sequences set forth in Table 4 below. In the context of the IgG1, the primary antibody has a molecular weight of about 143.5 kilodaltons.

TABLE 3
Nucleotide sequences of the variable
domains of a secondary anti-FRalpha antibody

| SEQ | | Sequence |
|---|---|---|
| ID | | 10 20 30 40 |
| NO. | Region | 12345678901234567890123456789012345678901234567890 |
| 5 | VH | GAGGTGCAGCTGTTGGACTCTGGGGGAGGCTTGGTCCAGCCTGGGAGGTC |
| | | CCTGAGACTCTCCTGTGCAGCGTCTGGATTCACCTTCAGTAGCTATAGCA |
| | | TGAACTGGGTCCGCCAGGCTCCAGGGAAGGGGCTGGAGTGGGTCTCATCC |
| | | ATTAGTAGTAGTAGTAGTTACATATACTACGCAGACTCAGTGAAGGGCCG |
| | | ATTCACCATCTCCAGAGACAACGCCAAGAACTCACTGTATCTGCAAATGA |
| | | ACAGCCTGAGAACTGAAGACACGGCCGTGTATTACTGTGCGAGACAAAGT |
| | | GGGAGCTTCGGGTACTTTGACTACTGGGGCCAAGGAACACCGGTCACCGT |
| | | CTCATCA |
| 6 | VL | GACATCCAGATGACCCAGTCTCCATCCTCCCTGTCTGCATCTGTAGGAGA |
| | | CAGAGTCACCATCACTTGCCGGGCCAGTCAGAATATTTTTACCTGGTTAG |
| | | CCTGGTATCAGCAGAAACCAGGGAAAGCCCCTAAACTCCTGATTTATGGT |
| | | GCATCCACTTTGCAGAGTGGGGTCCCATCAAGGTTCAGCGGCAGCGGATC |
| | | TGGGACAGACTTCACTCTCACCATCAGCAGTCTGCAGCCTGAAGATTTTG |
| | | CGACTTACTATTGTCAACAGGCTAAGAGTTTCCCTCTCACTTTCGGCGGA |
| | | GGGACCAAGGTGGAGATCAAA |

TABLE 4
Amino acid sequences of the variable
domains of a secondary anti-FRalpha antibody

| SEQ | | Sequence |
|---|---|---|
| ID | | 10 20 30 40 |
| NO. | Region | 12345678901234567890123456789012345678901234567890 |
| 7 | VH | EVQLLDSGGGLVQPGRSLRLSCAASGFTFSSYSMNWVRQAPGKGLEWVSS |
| | | ISSSSSYIYYADSVKGRFTISRDNAKNSLYLQMNSLRTEDTAVYYCARQS |
| | | GSFGYFDYWGQGTPVTVSS |
| 8 | VL | DIQMTQSPSSLSASVGDRVTITCRASQNIFTWLAWYQQKPGKAPKLLIYG |
| | | ASTLQSGVPSRFSGSGSGTDFTLTISSLQPEDFATYYCQQAKSFPLTFGG |
| | | GTKVEIK |

The nucleotide sequences of the human VH and VL regions of a secondary antibody, according to embodiments of the present disclosure, are set forth in Table 5 below. The variable regions have the amino acid sequences set forth in Table 6 below. In the context of the IgG1, the primary antibody has a molecular weight of about 143.5 kilodaltons.

TABLE 5
Nucleotide sequences of the variable
domains of a secondary anti-FRalpha antibody

| SEQ | | Sequence |
|---|---|---|
| ID | | 10 20 30 40 |
| NO. | Region | 12345678901234567890123456789012345678901234567890 |
| 9 | VH | GAGGTGCAGCTGTTGGAGTCTGGGGGAGGCGTGGTCCAGCCTGGGAGGTC |
| | | CCTGAGACTCTCCTGTGCAGCCTCTGGATTCACCTTCAGTAGCTATGGCA |
| | | TGCACTGGGTCCGCCAGGCTCCAGGCAAGGGGCTGGAGTGGGTGGCCAAC |
| | | ATAAAGGAAGATGGAAGTGAGAAATACTATGTGGACTCTGTGAAGGGCCG |
| | | GTTCACCATCTCCAGAGACAACGCCAAGAACTCACTGGATCTGCAGATGA |
| | | ACAGCCTGAGAGCCGAGGACACGGCTGTGTATTACTGTGCAAGAGGGGAA |
| | | CGCGATTTTTGGAGTGGTTCTTACTACTACGGTATGGACGTCTGGGGCCA |
| | | GGGGACCATGGTCACCGTCTCATCA |
| 10 | VL | AACATCCAGATGACCCAGTCTCCATCTTCCCTGTCTGCATCTGTAGGAGA |
| | | CACAGTCACCATCACTTGTCGGGCGAGTCAGGATGTTAGCAGGTGGTTAG |
| | | GCTGGTATCAGCAGAAACCAGGCAAAGCCCCTCGGCTCCTGATCTCTGCT |
| | | ACATCCCGTTTGCAAAGTGGTGTCCCATCAAGGTTCAGCGGCAGTGGATC |
| | | TGGGACAGAATTCACTCTCATCATCAGCAACCTGCAGCCGGAAGATTTTG |
| | | CGACTTACTACTGTCAACAGGTCAACAACTTCCCGATCACCTTCGGCGGA |
| | | GGGACCAAGGTGGAGATCAAG |

TABLE 6
Amino acid sequences of the variable
domains of a secondary anti-FRalpha antibody

| SEQ | | Sequence |
|---|---|---|
| ID | | 10 20 30 40 |
| NO. | Region | 12345678901234567890123456789012345678901234567890 |
| 11 | VH | EVQLLESGGGVVQPGRSLRLSCAASGFTFSSYGMHWVRQAPGKGLEWVAN |
| | | IKEDGSEKYYVDSVKGRFTISRDNAKNSLDLQMNSLRAEDTAVYYCARGE |
| | | RDFWSGSYYYGMDVWGQGTMVTVSS |
| 12 | VL | NIQMTQSPSSLSASVGDTVTITCRASQDVSRWLGWYQQKPGKAPRLLISA |
| | | TSRLQSGVPSRFSGSGSGTEFTLIISNLQPEDFATYYCQQVNNFPITFGG |
| | | GTKVEIK |

The nucleotide sequences of the human VH and VL regions of a secondary antibody, according to embodiments of the present disclosure, are set forth in Table 7 below. The variable regions have the amino acid sequences set forth in Table 8 below. In the context of the IgG1, the primary antibody has a molecular weight of about 143.5 kilodaltons.

TABLE 7
Nucleotide sequences of the variable
domains of a secondary anti-FRalpha antibody

| SEQ | | Sequence |
|---|---|---|
| ID | | 10 20 30 40 |
| NO. | Region | 12345678901234567890123456789012345678901234567890 |
| 13 | VH | CAGGTGCAGCTGTTGGAGTCTGGGGGAGGCCTGGTCAAGCCTGGGGGGTC |
| | | CCTGAGACTCTCCTGTGCAGCCTCTGGATTCACCTTCAGTAGCTATAGCA |
| | | TGAACTGGGTCCGCCAGGCTCCAGGGAAGGGGCTGGAGTGGGTCTCATCC |
| | | ATTAGTAGTAGTAGTAGTTACATATACTACGCAGACTCAGTGAAGGGCCG |
| | | ATTCACCATCTCCAGAGACAACGCCAAGAACTCACTGTATCTGCAAATGA |
| | | ACAGCCTGAGAGCCGAGGACACGGCTGTGTATTACTGTGCGAGAGCAGCA |
| | | ACAACTGGTACGGTTGACTACTGGGGCCAGGGAACAACGGTCACCGTCTC |
| | | ATCA |
| 14 | VL | GACATCCAGATGACCCAGTCTCCATCTTCCGTGTCTGCATCTGTGGGAGA |
| | | CAGAGTCACCATTACTTGTCGGGCGAGTCAGGGCATTAGCACCTGGTTAG |
| | | CCTGGTATCAGCAGAAACCAGGGAAAGCCCCTAAACTCCTCATCTATGCT |
| | | GCATCCAGTTTGCAAAATGGGGTCCCATCAAGGTTCAGCGGCAGTGGATT |
| | | TGGGACAGATTTCACTCTCACCATCAGCAGCCTGCAGCCTGAAGACTTTG |
| | | CAACTTACTATTGTCAACAGGCTAGCAGTTTCCCTCTCACTTTCGGCGGA |
| | | GGGACCAAGGTGGAGATCAAA |

TABLE 8
Amino acid sequences of the variable
domains of a secondary anti-FRalpha antibody

| SEQ | | Sequence |
|---|---|---|
| ID | | 10 20 30 40 |
| NO. | Region | 12345678901234567890123456789012345678901234567890 |
| 15 | VH | QVQLLESGGGLVKPGGSLRLSCAASGFTFSSYSMNWVRQAPGKGLEWVSS |
| | | ISSSSSYIYYADSVKGRFTISRDNAKNSLYLQMNSLRAEDTAVYYCARAA |
| | | TTGTVDYWGQGTTVTVSS |
| 16 | VL | DIQMTQSPSSVSASVGDRVTITCRASQGISTWLAWYQQKPGKAPKLLIYA |
| | | ASSLQNGVPSRFSGSGFGTDFTLTISSLQPEDFATYYCQQASSFPLTFGG |
| | | GTKVEIK |

In the context of the IgG1, the nucleotide sequences of human full heavy chains of a secondary antibody, according to embodiments of the present disclosure, are set forth in Table 9 below and the corresponding variable heavy chains are set forth in Table 11. That is, SEQ ID NO: 24 is the variable region of SEQ ID NO: 17, SEQ ID NO: 25 is the variable region of SEQ ID NO: 18, and SEQ ID NO: 26 is the variable region of SEQ ID NO: 19.

The nucleotide sequences of human full light chains of a secondary antibody, according to embodiments of the present disclosure, are set forth in Table 10 below and the corresponding variable light chains are set forth in Table 12. That is, SEQ ID NO: 27 is the variable region of SEQ ID NO: 20, SEQ ID NO: 28 is the variable region of SEQ ID NO: 21, SEQ ID NO: 29 is the variable region of SEQ ID NO: 22, SEQ ID NO: 30 is the variable region of SEQ ID NO: 23.

The amino acid sequences of human full heavy chains of a secondary antibody, according to embodiments of the present disclosure, are set forth in Table 13, below, and the corresponding variable heavy chains are set forth in Table 15. That is, SEQ ID NO: 38 is the variable region of SEQ ID NO: 31, SEQ ID NO: 39 is the variable region of SEQ ID NO: 32, and SEQ ID NO: 40 is the variable region of SEQ ID NO: 33.

The amino acid sequences of human full light chains of a secondary antibody, according to embodiments of the present disclosure, are set forth in Table 14, below, and the corresponding variable light chains are set forth in Table 16. That is, SEQ ID NO: 41 is the variable region of SEQ ID NO: 34, SEQ ID NO: 42 is the variable region of SEQ ID NO: 35, SEQ ID NO: 43 is the variable region of SEQ ID NO: 36, and SEQ ID NO: 44 is the variable region of SEQ ID NO: 37.

Various aspects of this disclosure relate to at least a pair of antibodies, where the secondary antibody comprises a full heavy chain nucleotide sequence according to SEQUENCE ID. NO: 17, 18, or 19. In some embodiments, the secondary antibody comprises a nucleotide sequence that is at least 70 percent identical to SEQ ID NO: 17, 18, or 19. In some specific embodiments, the secondary antibody comprises a nucleotide sequence that is at least 80 percent identical to SEQ ID NO: 17, 18, or 19. In some even more specific embodiments, the secondary antibody comprises a nucleotide sequence that is at least 90 percent identical to SEQ ID NO: 17, 18, or 19. In some very specific embodiments, the secondary antibody comprises a nucleotide sequence that is at least 95 percent identical to SEQ ID NO: 17, 18, or 19.

Various aspects of this disclosure relate to at least a pair of antibodies, where the secondary antibody comprises a full light chain nucleotide sequence according to SEQUENCE ID. NO: 20, 21, 22, or 23. In some embodiments, the secondary antibody comprises a nucleotide sequence that is at least 70 percent identical to SEQ ID NO: 20, 21, 22, or 23. In some specific embodiments, the secondary antibody comprises a nucleotide sequence that is at least 80 percent identical to SEQ ID NO: 20, 21, 22, or 23. In some even more specific embodiments, the secondary antibody comprises a nucleotide sequence that is at least 90 percent identical to SEQ ID NO: 20, 21, 22, or 23. In some very specific embodiments, the secondary antibody comprises a nucleotide sequence that is at least 95 percent identical to SEQ ID NO: 20, 21, 22, or 23.

Various aspects of this disclosure relate to at least a pair of antibodies, where the secondary antibody comprises a variable heavy chain nucleotide sequence according to SEQUENCE ID. NO: 24, 25, or 26. In some embodiments, the secondary antibody comprises a nucleotide sequence that is at least 70 percent identical to SEQ ID NO: 24, 25, or 26. In some specific embodiments, the secondary antibody comprises a nucleotide sequence that is at least 80 percent identical to SEQ ID NO: 24, 25, or 26. In some even more specific embodiments, the secondary antibody comprises a nucleotide sequence that is at least 90 percent identical to SEQ ID NO: 24, 25, or 26. In some very specific embodiments, the secondary antibody comprises a nucleotide sequence that is at least 95 percent identical to SEQ ID NO: 24, 25, or 26.

Various aspects of this disclosure relate to at least a pair of antibodies, where the secondary antibody comprises a variable light chain nucleotide sequence according to SEQUENCE ID. NO: 27, 28, 29, or 30. In some embodiments, the secondary antibody comprises a nucleotide sequence that is at least 70 percent identical to SEQ ID NO: 27, 28, 29, or 30. In some specific embodiments, the secondary antibody comprises a nucleotide sequence that is at least 80 percent identical to SEQ ID NO: 27, 28, 29, or 30. In some even more specific embodiments, the secondary antibody comprises a nucleotide sequence that is at least 90 percent identical to SEQ ID NO: 27, 28, 29, or 30. In some very specific embodiments, the secondary antibody comprises a nucleotide sequence that is at least 95 percent identical to SEQ ID NO: 27, 28, 29, or 30.

Various aspects of this disclosure relate to at least a pair of antibodies, where the secondary antibody comprises a full heavy chain amino acid sequence according to SEQUENCE ID. NO: 31, 32, or 33. In some embodiments, the secondary antibody comprises an amino acid sequence that is at least 70 percent identical to SEQ ID NO: 31, 32, or 33. In some specific embodiments, the secondary antibody comprises a nucleotide sequence that is at least 80 percent identical to SEQ ID NO: 31, 32, or 33. In some even more specific embodiments, the secondary antibody comprises a nucleotide sequence that is at least 90 percent identical to SEQ ID NO: 31, 32, or 33. In some very specific embodiments, the secondary antibody comprises a nucleotide sequence that is at least 95 percent identical to SEQ ID NO: 31, 32, or 33.

Various aspects of this disclosure relate to at least a pair of antibodies, where the secondary antibody comprises a full light chain amino acid sequence according to SEQUENCE ID. NO: 34, 35, 36, or 37. In some embodiments, the secondary antibody comprises an amino acid sequence that is at least 70 percent identical to SEQ ID NO: 34, 35, 36, or 37. In some specific embodiments, the secondary antibody comprises a nucleotide sequence that is at least 80 percent identical to SEQ ID NO: 34, 35, 36, or 37. In some even more specific embodiments, the secondary antibody comprises a nucleotide sequence that is at least 90 percent identical to SEQ ID NO: 34, 35, 36, or 37. In some very specific embodiments, the secondary antibody comprises a nucleotide sequence that is at least 95 percent identical to SEQ ID NO: 34, 35, 36, or 37.

Various aspects of this disclosure relate to at least a pair of antibodies, where the secondary antibody comprises a variable heavy chain amino acid sequence according to SEQUENCE ID. NO: 38, 39, or 40. In some embodiments, the secondary antibody comprises an amino acid sequence that is at least 70 percent identical to SEQ ID NO: 38, 39, or 40. In some specific embodiments, the secondary antibody comprises a nucleotide sequence that is at least 80 percent identical to SEQ ID NO: 38, 39, or 40. In some even more specific embodiments, the secondary antibody comprises a nucleotide sequence that is at least 90 percent identical to SEQ ID NO: 38, 39, or 40. In some very specific embodiments, the secondary antibody comprises a nucleotide sequence that is at least 95 percent identical to SEQ ID NO: 38, 39, or 40.

Various aspects of this disclosure relate to at least a pair of antibodies, where the secondary antibody comprises a variable light chain amino acid sequence according to SEQUENCE ID. NO: 41, 42, 43, or 44. In some embodiments, the secondary antibody comprises an amino acid sequence that is at least 70 percent identical to SEQ ID NO: 41, 42, 43, or 44. In some specific embodiments, the secondary antibody comprises a nucleotide sequence that is at least 80 percent identical to SEQ ID NO: 41, 42, 43, or 44. In some even more specific embodiments, the secondary antibody comprises a nucleotide sequence that is at least 90 percent identical to SEQ ID NO: 41, 42, 43, or 44. In some very specific embodiments, the secondary antibody comprises a nucleotide sequence that is at least 95 percent identical to SEQ ID NO: 41, 42, 43, or 44.

In a non-limiting example, a secondary antibody of the present disclosure comprises a full heavy chain according to the nucleotide sequence of SEQ ID NO: 18 and a full light chain according to the nucleotide sequence of SEQ ID NO: 21. The secondary antibody, of the non-limiting example comprises a full heavy chain according to the amino acid sequence of SEQ ID NO: 32 and a full light chain according to the amino acid sequence of SEQ ID NO: 35. In the context of the IgG1, the secondary antibody has a molecular weight of about 143.5 kilodaltons.

In a non-limiting example, a secondary antibody of the present disclosure comprises a full heavy chain according to the nucleotide sequence of SEQ ID NO: 17 and a full light chain according to the nucleotide sequence of SEQ ID NO: 22. The secondary antibody, of the non-limiting example comprises a full heavy chain according to the amino acid sequence of SEQ ID NO: 31 and a full light chain according to the amino acid sequence of SEQ ID NO: 36. In the context of the IgG1, the secondary antibody has a molecular weight of about 143.5 kilodaltons.

In a non-limiting example, a secondary antibody of the present disclosure comprises a full heavy chain according to the nucleotide sequence of SEQ ID NO: 19 and a full light chain according to the nucleotide sequence of SEQ ID NO: 20. The secondary antibody, of the non-limiting example comprises a full heavy chain according to the amino acid sequence of SEQ ID NO: 33 and a full light chain according to the amino acid sequence of SEQ ID NO: 34. In the context of the IgG1, the secondary antibody has a molecular weight of about 143.5 kilodaltons.

In a non-limiting example, a secondary antibody of the present disclosure comprises a full heavy chain according to the nucleotide sequence of SEQ ID NO: 17 and a full light chain according to the nucleotide sequence of SEQ ID NO: 23. The secondary antibody, of the non-limiting example comprises a full heavy chain according to the amino acid sequence of SEQ ID NO: 31 and a full light chain according to the amino acid sequence of SEQ ID NO: 37. In the context of the IgG1, the secondary antibody has a molecular weight of about 143.5 kilodaltons.

The affinity of the antibodies associated with SEQ ID NO: 17-44 for FRalpha was determined by surface plasmon resonance (SPR) and the inhibition rates between the antibodies associated with SEQ ID NO: 17-44 were determined by biolayer inferometric (BLI) principle, as described in more detail with reference to Example 6.

TABLE 9
Nucleotide sequences of full heavy
chains a secondary anti-FRalpha antibody

| SEQ | | Sequence |
|---|---|---|
| ID | | 10 20 30 40 |
| NO. | Region | 12345678901234567890123456789012345678901234567890 |
| 17 | Full | GAGGTGCAGCTGTTGGAGTCTGGGGGAGGCGTGGTCCAGCCTGGGAGGTC |
| | Heavy | CCTGAGACTCTCCTGTGCAGCCTCTGGATTCATGTTTAGTAACTATTGGA |
| | Chain | TGATTTGGGTCCGTCAGGCTGCAGGGAAGGGTCCTGAGTGGGTGGCAGTT |
| | | ATATCATATGATGGAAGCAATAAATTGTACGCAGACTCCGTGAAGGGCCG |
| | | ATTCACCATCTCCAGAGACAATTCCAAGAACACGCTGTATCTGCAAATGA |
| | | ACAGCCTGAGAGCTGAGGACACTGCCGTGTATTACTGTGCGAGAGTACCA |
| | | GTAGGTTACTACTACTACTACGGTATGGACGTCTGGGGTCAGGGGACTCC |
| | | GGTCACCGTCTCATCAGCTTCCACCAAGGGCCCCTCCGTGTTCCCCCTGG |
| | | CTCCCTCTTCCAAGAGCACCAGCGGCGGCACCGCTGCTCTGGGATGTCTG |
| | | GTGAAGGACTACTTCCCTGAGCCTGTGACCGTGTCCTGGAATTCCGGCGC |
| | | CCTGACCTCCGGCGTGCACACATTCCCTGCTGTGCTGCAGTCCTCCGGCC |
| | | TGTATAGCCTGTCCTCCGTGGTGACAGTGCCTAGCTCCAGCCTGGGCACC |
| | | CAGACCTATATCTGCAACGTGAACCACAAGCCTAGCAATACCAAGGTGGA |
| | | CAAGAAGGTGGAGCCTAAGAGCTGCGACAAGACCCACACCTGTCCTCCAT |
| | | GTCCTGCTCCAGAACTGCTCGGCGGACCTTCCGTGTTCCTGTTTCCTCCA |
| | | AAGCCTAAGGACACCCTGATGATCAGCAGAACCCCTGAAGTGACCTGCGT |
| | | GGTGGTGGATGTGTCCCACGAGGATCCCGAAGTGAAGTTCAATTGGTACG |
| | | TGGACGGCGTGGAAGTGCACAACGCCAAGACCAAGCCTAGAGAGGAACAG |
| | | TACAACAGCACCTACAGAGTGGTGTCCGTGCTGACCGTGCTGCACCAGGA |
| | | TTGGCTGAACGGCAAAGAGTACAAGTGCAAGGTGTCCAACAAGGCCCTGC |
| | | CTGCTCCTATCGAGAAAACCATCAGCAAGGCCAAGGGCCAGCCTAGGGAA |
| | | CCCCAGGTTTACACACTGCCTCCAAGCAGGGACGAGCTGACCAAGAATCA |
| | | GGTGTCCCTGACCTGCCTGGTCAAGGGCTTCTACCCTTCCGATATCGCCG |
| | | TGGAATGGGAGAGCAATGGCCAGCCTGAGAACAACTACAAGACAACCCCT |
| | | CCTGTGCTGGACAGCGACGGCTCATTCTTCCTGTACAGCAAGCTGACAGT |
| | | GGACAAGAGCAGATGGCAGCAGGGCAACGTGTTCAGCTGCAGCGTGATGC |
| | | ACGAGGCCCTGCACAACCACTACACCCAGAAGTCCCTGAGCCTGTCTCCT |
| | | GGCAAA |
| 18 | Full | GAGGTGCAGCTGTTGGAGTCTGGGGGAGGCGTGGTCCAGCCTGGGAGGTC |
| | Heavy | CCTGAGACTCTCCTGTGCAGCCTCTGGATTCATGTTTAGTAACTATTGGA |
| | Chain | TGATTTGGGTCCGTCAGGCTGCAGGGAAGGGTCCTGAGTGGGTGGCAGTT |
| | | ATATCATATGATGGAAGCAATAAATTGTACGCAGACTCCGTGAAGGGCCG |
| | | ATTCACCATCTCCAGAGACAATTCCAAGAACACGCTGTATCTGCAAATGA |
| | | ACAGCCTGAGAGCTGAGGACACTGCCGTGTATTACTGTGCGAGAGTACCA |
| | | GTAGGTTACTACTACTACTACTCGATGGACGTCTGGGGTCAGGGGACTCC |
| | | GGTCACCGTCTCATCAGCTTCCACCAAGGGCCCCTCCGTGTTCCCCCTGG |
| | | CTCCCTCTTCCAAGAGCACCAGCGGCGGCACCGCTGCTCTGGGATGTCTG |
| | | GTGAAGGACTACTTCCCTGAGCCTGTGACCGTGTCCTGGAATTCCGGCGC |
| | | CCTGACCTCCGGCGTGCACACATTCCCTGCTGTGCTGCAGTCCTCCGGCC |
| | | TGTATAGCCTGTCCTCCGTGGTGACAGTGCCTAGCTCCAGCCTGGGCACC |
| | | CAGACCTATATCTGCAACGTGAACCACAAGCCTAGCAATACCAAGGTGGA |
| | | CAAGAAGGTGGAGCCTAAGAGCTGCGACAAGACCCACACCTGTCCTCCAT |
| | | GTCCTGCTCCAGAACTGCTCGGCGGACCTTCCGTGTTCCTGTTTCCTCCA |
| | | AAGCCTAAGGACACCCTGATGATCAGCAGAACCCCTGAAGTGACCTGCGT |
| | | GGTGGTGGATGTGTCCCACGAGGATCCCGAAGTGAAGTTCAATTGGTACG |
| | | TGGACGGCGTGGAAGTGCACAACGCCAAGACCAAGCCTAGAGAGGAACAG |
| | | TACAACAGCACCTACAGAGTGGTGTCCGTGCTGACCGTGCTGCACCAGGA |
| | | TTGGCTGAACGGCAAAGAGTACAAGTGCAAGGTGTCCAACAAGGCCCTGC |
| | | CTGCTCCTATCGAGAAAACCATCAGCAAGGCCAAGGGCCAGCCTAGGGAA |
| | | CCCCAGGTTTACACACTGCCTCCAAGCAGGGACGAGCTGACCAAGAATCA |
| | | GGTGTCCCTGACCTGCCTGGTCAAGGGCTTCTACCCTTCCGATATCGCCG |
| | | TGGAATGGGAGAGCAATGGCCAGCCTGAGAACAACTACAAGACAACCCCT |
| | | CCTGTGCTGGACAGCGACGGCTCATTCTTCCTGTACAGCAAGCTGACAGT |
| | | GGACAAGAGCAGATGGCAGCAGGGCAACGTGTTCAGCTGCAGCGTGATGC |
| | | ACGAGGCCCTGCACAACCACTACACCCAGAAGTCCCTGAGCCTGTCTCCT |
| | | GGCAAA |
| 19 | Full | GAGGTGCAGCTGTTGGAGTCTGGGGGAGGCGTGGTCCAGCCTGGGAGGTC |
| | Heavy | CCTGAGACTCTCCTGTGCAGCCTCTGGATTCATGTTTAGTAACTATTGGA |
| | Chain | TGATTTGGGTCCGTCAGGCTGCAGGGAAGGGTCCTGAGTGGGTGGCAGTT |
| | | ATATCATATGATGGAAGCAATAAATTGTACGCAGACTCCGTGAAGGGCCG |
| | | ATTCACCATCTCCAGAGACAATTCCAAGAACACGCTGTATCTGCAAATGA |
| | | ACAGCCTGAGAGCTGAGGACACTGCCGTGTATTACTGTGCGAGAGTACCA |
| | | ATTGCTTACTACTACTACTACGGTATGGACGTCTGGGGTCAGGGGACTCC |
| | | GGTCACCGTCTCATCAGCTTCCACCAAGGGCCCCTCCGTGTTCCCCCTGG |
| | | CTCCCTCTTCCAAGAGCACCAGCGGCGGCACCGCTGCTCTGGGATGTCTG |
| | | GTGAAGGACTACTTCCCTGAGCCTGTGACCGTGTCCTGGAATTCCGGCGC |
| | | CCTGACCTCCGGCGTGCACACATTCCCTGCTGTGCTGCAGTCCTCCGGCC |
| | | TGTATAGCCTGTCCTCCGTGGTGACAGTGCCTAGCTCCAGCCTGGGCACC |
| | | CAGACCTATATCTGCAACGTGAACCACAAGCCTAGCAATACCAAGGTGGA |
| | | CAAGAAGGTGGAGCCTAAGAGCTGCGACAAGACCCACACCTGTCCTCCAT |
| | | GTCCTGCTCCAGAACTGCTCGGCGGACCTTCCGTGTTCCTGTTTCCTCCA |
| | | AAGCCTAAGGACACCCTGATGATCAGCAGAACCCCTGAAGTGACCTGCGT |
| | | GGTGGTGGATGTGTCCCACGAGGATCCCGAAGTGAAGTTCAATTGGTACG |
| | | TGGACGGCGTGGAAGTGCACAACGCCAAGACCAAGCCTAGAGAGGAACAG |
| | | TACAACAGCACCTACAGAGTGGTGTCCGTGCTGACCGTGCTGCACCAGGA |
| | | TTGGCTGAACGGCAAAGAGTACAAGTGCAAGGTGTCCAACAAGGCCCTGC |
| | | CTGCTCCTATCGAGAAAACCATCAGCAAGGCCAAGGGCCAGCCTAGGGAA |
| | | CCCCAGGTTTACACACTGCCTCCAAGCAGGGACGAGCTGACCAAGAATCA |
| | | GGTGTCCCTGACCTGCCTGGTCAAGGGCTTCTACCCTTCCGATATCGCCG |
| | | TGGAATGGGAGAGCAATGGCCAGCCTGAGAACAACTACAAGACAACCCCT |
| | | CCTGTGCTGGACAGCGACGGCTCATTCTTCCTGTACAGCAAGCTGACAGT |
| | | GGACAAGAGCAGATGGCAGCAGGGCAACGTGTTCAGCTGCAGCGTGATGC |
| | | ACGAGGCCCTGCACAACCACTACACCCAGAAGTCCCTGAGCCTGTCTCCT |
| | | GGCAAA |

TABLE 10
Nucleotide sequences of full light chains
a secondary anti-FRalpha antibody

| SEQ | | Sequence |
|---|---|---|
| ID | | 10 20 30 40 |
| NO. | Region | 12345678901234567890123456789012345678901234567890 |
| 20 | Full | GCCATCCGGTTGACCCAGTCTCCATCCTTCCTGTCTGCATCTGTAGGAGA |
| | Light | CAGAGTCACCATCACTTGTCGTGCCAGTCAGGGCATTAGGAATTATTTAG |
| | Chain | CCTGGTATCAGCAAAAACCAGGGAAAGCTCCTAACCTCCTGATCTATGCT |
| | | GCATCCTTGTTGCAAAGTGGGGTCCCATCAAGGTTCAGCGGCAGTGGATT |
| | | TGGGACAGATTTCACTCTCACCATCAGCAGCCTGCAGCCTGAAGACTTTG |
| | | CAACTTACTATTGTCAACAGGCTAGCAGTTTCCCTCTCACTTTCGGTGGA |
| | | GGGACCAAGGTGGAGATCAAAAGGACCGTGGCTGCCCCCAGCGTGTTCAT |
| | | CTTCCCTCCTAGCGACGAGCAGCTGAAGAGCGGCACCGCTAGCGTGGTGT |
| | | GTCTGCTGAATAACTTCTATCCCAGGGAGGCCAAGGTGCAGTGGAAGGTG |
| | | GATAACGCCCTGCAGAGCGGCAACTCCCAGGAGTCCGTGACCGAGCAGGA |
| | | CTCCAAGGACAGCACCTACTCCCTGAGCTCCACCCTGACCCTGTCCAAGG |
| | | CTGATTATGAGAAGCACAAGGTGTATGCTTGCGAGGTGACACACCAGGGC |
| | | CTGTCCAGCCCTGTGACCAAGAGCTTCAACCGGGGCGAGTGC |
| 21 | Full | GCCATCCGGTTGACCCAGTCTCCATCCTTCCTGTCTGCATCTGTAGGAGA |
| | Light | CAGAGTCACCATCACTTGTCGTGCCAGTCAGGGCATTAGGAATTATTTTG |
| | Chain | CCTGGTATCAGCAAAAACCAGGGAAAGCTCCTAACCTCCTGATCTATGCT |
| | | GCATCCTTGTTGCAAAGTGGGGTCCCATCAAGGTTCAGCGGCAGTGGATT |
| | | TGGGACAGATTTCACTCTCACCATCAGCAGCCTGCAGCCTGAAGACTTTG |
| | | CAACTTACTATTGTCAACAGGCTAGCAGTTTCCCTTTTACTTTCGGTGGA |
| | | GGGACCAAGGTGGAGATCAAAAGGACCGTGGCTGCCCCCAGCGTGTTCAT |
| | | CTTCCCTCCTAGCGACGAGCAGCTGAAGAGCGGCACCGCTAGCGTGGTGT |
| | | GTCTGCTGAATAACTTCTATCCCAGGGAGGCCAAGGTGCAGTGGAAGGTG |
| | | GATAACGCCCTGCAGAGCGGCAACTCCCAGGAGTCCGTGACCGAGCAGGA |
| | | CTCCAAGGACAGCACCTACTCCCTGAGCTCCACCCTGACCCTGTCCAAGG |
| | | CTGATTATGAGAAGCACAAGGTGTATGCTTGCGAGGTGACACACCAGGGC |
| | | CTGTCCAGCCCTGTGACCAAGAGCTTCAACCGGGGCGAGTGC |
| 22 | Full | GCCATCCGGTTGACCCAGTCTCCATCCTTCCTGTCTGCATCTGTAGGAGA |
| | Light | CAGAGTCACCATCACTTGTCGTGCCAGTCAGGGCATTAGGAATTATTTAG |
| | Chain | CCTGGTATCAGCAAAAACCAGGGAAAGCTCCTAACCTCCTGATCTATGCT |
| | | GCATCCTTGTTGCAAAGTGGGGTCCCATCAAGGTTCAGCGGCAGTGGATT |
| | | TGGGACAGATTTCACTCTCACCATCAGCAGCCTGCAGCCTGAAGACTTTG |
| | | CAACTTACTATTGTCAAGATGCTAGCAGTTTCCCTCTCACTTTCGGTGGA |
| | | GGGACCAAGGTGGAGATCAAAAGGACCGTGGCTGCCCCCAGCGTGTTCAT |
| | | CTTCCCTCCTAGCGACGAGCAGCTGAAGAGCGGCACCGCTAGCGTGGTGT |
| | | GTCTGCTGAATAACTTCTATCCCAGGGAGGCCAAGGTGCAGTGGAAGGTG |
| | | GATAACGCCCTGCAGAGCGGCAACTCCCAGGAGTCCGTGACCGAGCAGGA |
| | | CTCCAAGGACAGCACCTACTCCCTGAGCTCCACCCTGACCCTGTCCAAGG |
| | | CTGATTATGAGAAGCACAAGGTGTATGCTTGCGAGGTGACACACCAGGGC |
| | | CTGTCCAGCCCTGTGACCAAGAGCTTCAACCGGGGCGAGTGC |
| 23 | Full | GCCATCCGGTTGACCCAGTCTCCATCCTTCCTGTCTGCATCTGTAGGAGA |
| | Light | CAGAGTCACCATCACTTGTCGTGCCAGTCAGGGCATTAGGAATTATTTAG |
| | Chain | CCTGGTATCAGCAAAAACCAGGGAAAGCTCCTAACCTCCTGATCTATGCT |
| | | GCATCCTTGTTGCAAAGTGGGGTCCCATCAAGGTTCAGCGGCAGTGGATT |
| | | TGGGACAGATTTCACTCTCACCATCAGCAGCCTGCAGCCTGAAGACTTTG |
| | | CAACTTACTATTGTCAACAGGCTAGCAGTTTCCCTGTTCATTTCGGTGGA |
| | | GGGACCAAGGTGGAGATCAAAAGGACCGTGGCTGCCCCCAGCGTGTTCAT |
| | | CTTCCCTCCTAGCGACGAGCAGCTGAAGAGCGGCACCGCTAGCGTGGTGT |
| | | GTCTGCTGAATAACTTCTATCCCAGGGAGGCCAAGGTGCAGTGGAAGGTG |
| | | GATAACGCCCTGCAGAGCGGCAACTCCCAGGAGTCCGTGACCGAGCAGGA |
| | | CTCCAAGGACAGCACCTACTCCCTGAGCTCCACCCTGACCCTGTCCAAGG |
| | | CTGATTATGAGAAGCACAAGGTGTATGCTTGCGAGGTGACACACCAGGGC |
| | | CTGTCCAGCCCTGTGACCAAGAGCTTCAACCGGGGCGAGTGC |

TABLE 11
Nucleotide sequences of variable heavy
chains of a secondary anti-FRalpha antibody

| SEQ | | Sequence |
|---|---|---|
| ID | | 10 20 30 40 |
| NO. | Region | 12345678901234567890123456789012345678901234567890 |
| 24 | VH | GAGGTGCAGCTGTTGGAGTCTGGGGGAGGCGTGGTCCAGCCTGGGAGGTC |
| | | CCTGAGACTCTCCTGTGCAGCCTCTGGATTCATGTTTAGTAACTATTGGA |
| | | TGATTTGGGTCCGTCAGGCTGCAGGGAAGGGTCCTGAGTGGGTGGCAGTT |
| | | ATATCATATGATGGAAGCAATAAATTGTACGCAGACTCCGTGAAGGGCCG |
| | | ATTCACCATCTCCAGAGACAATTCCAAGAACACGCTGTATCTGCAAATGA |
| | | ACAGCCTGAGAGCTGAGGACACTGCCGTGTATTACTGTGCGAGAGTACCA |
| | | GTAGGTTACTACTACTACTACGGTATGGACGTCTGGGGTCAGGGGACTCC |
| | | GGTCACCGTCTCATCA |
| 25 | VH | GAGGTGCAGCTGTTGGAGTCTGGGGGAGGCGTGGTCCAGCCTGGGAGGTC |
| | | CCTGAGACTCTCCTGTGCAGCCTCTGGATTCATGTTTAGTAACTATTGGA |
| | | TGATTTGGGTCCGTCAGGCTGCAGGGAAGGGTCCTGAGTGGGTGGCAGTT |
| | | ATATCATATGATGGAAGCAATAAATTGTACGCAGACTCCGTGAAGGGCCG |
| | | ATTCACCATCTCCAGAGACAATTCCAAGAACACGCTGTATCTGCAAATGA |
| | | ACAGCCTGAGAGCTGAGGACACTGCCGTGTATTACTGTGCGAGAGTACCA |
| | | GTAGGTTACTACTACTACTACTCGATGGACGTCTGGGGTCAGGGGACTCC |
| | | GGTCACCGTCTCATCA |
| 26 | VH | GAGGTGCAGCTGTTGGAGTCTGGGGGAGGCGTGGTCCAGCCTGGGAGGTC |
| | | CCTGAGACTCTCCTGTGCAGCCTCTGGATTCATGTTTAGTAACTATTGGA |
| | | TGATTTGGGTCCGTCAGGCTGCAGGGAAGGGTCCTGAGTGGGTGGCAGTT |
| | | ATATCATATGATGGAAGCAATAAATTGTACGCAGACTCCGTGAAGGGCCG |
| | | ATTCACCATCTCCAGAGACAATTCCAAGAACACGCTGTATCTGCAAATGA |
| | | ACAGCCTGAGAGCTGAGGACACTGCCGTGTATTACTGTGCGAGAGTACCA |
| | | ATTGCTTACTACTACTACTACGGTATGGACGTCTGGGGTCAGGGGACTCC |
| | | GGTCACCGTCTCATCA |

TABLE 12
Nucleotide sequences of variable light
chains of a secondary anti-FRalpha antibody

| SEQ | | Sequence |
|---|---|---|
| ID | | 10 20 30 40 |
| NO. | Region | 12345678901234567890123456789012345678901234567890 |
| 27 | VL | GCCATCCGGTTGACCCAGTCTCCATCCTTCCTGTCTGCATCTGTAGGAGA |
| | | CAGAGTCACCATCACTTGTCGTGCCAGTCAGGGCATTAGGAATTATTTAG |
| | | CCTGGTATCAGCAAAAACCAGGGAAAGCTCCTAACCTCCTGATCTATGCT |
| | | GCATCCTTGTTGCAAAGTGGGGTCCCATCAAGGTTCAGCGGCAGTGGATT |
| | | TGGGACAGATTTCACTCTCACCATCAGCAGCCTGCAGCCTGAAGACTTTG |
| | | CAACTTACTATTGTCAACAGGCTAGCAGTTTCCCTCTCACTTTCGGTGGA |
| | | GGGACCAAGGTGGAGATCAAA |
| 28 | VL | GCCATCCGGTTGACCCAGTCTCCATCCTTCCTGTCTGCATCTGTAGGAGA |
| | | CAGAGTCACCATCACTTGTCGTGCCAGTCAGGGCATTAGGAATTATTTTG |
| | | CCTGGTATCAGCAAAAACCAGGGAAAGCTCCTAACCTCCTGATCTATGCT |
| | | GCATCCTTGTTGCAAAGTGGGGTCCCATCAAGGTTCAGCGGCAGTGGATT |
| | | TGGGACAGATTTCACTCTCACCATCAGCAGCCTGCAGCCTGAAGACTTTG |
| | | CAACTTACTATTGTCAACAGGCTAGCAGTTTCCCTTTTACTTTCGGTGGA |
| | | GGGACCAAGGTGGAGATCAAA |
| 29 | VL | GCCATCCGGTTGACCCAGTCTCCATCCTTCCTGTCTGCATCTGTAGGAGA |
| | | CAGAGTCACCATCACTTGTCGTGCCAGTCAGGGCATTAGGAATTATTTAG |
| | | CCTGGTATCAGCAAAAACCAGGGAAAGCTCCTAACCTCCTGATCTATGCT |
| | | GCATCCTTGTTGCAAAGTGGGGTCCCATCAAGGTTCAGCGGCAGTGGATT |
| | | TGGGACAGATTTCACTCTCACCATCAGCAGCCTGCAGCCTGAAGACTTTG |
| | | CAACTTACTATTGTCAAGATGCTAGCAGTTTCCCTCTCACTTTCGGTGGA |
| | | GGGACCAAGGTGGAGATCAAA |
| 30 | VL | GCCATCCGGTTGACCCAGTCTCCATCCTTCCTGTCTGCATCTGTAGGAGA |
| | | CAGAGTCACCATCACTTGTCGTGCCAGTCAGGGCATTAGGAATTATTTAG |
| | | CCTGGTATCAGCAAAAACCAGGGAAAGCTCCTAACCTCCTGATCTATGCT |
| | | GCATCCTTGTTGCAAAGTGGGGTCCCATCAAGGTTCAGCGGCAGTGGATT |
| | | TGGGACAGATTTCACTCTCACCATCAGCAGCCTGCAGCCTGAAGACTTTG |
| | | CAACTTACTATTGTCAACAGGCTAGCAGTTTCCCTGTTCATTTCGGTGGA |
| | | GGGACCAAGGTGGAGATCAAA |

TABLE 13
Amino Acid sequences of full heavy
chains of a secondary anti-FRalpha antibody

| SEQ | | Sequence |
|---|---|---|
| ID | | 10 20 30 40 |
| NO. | Region | 12345678901234567890123456789012345678901234567890 |
| 31 | Full | EVQLLESGGGVVQPGRSLRLSCAASGFMFSNYWMIWVRQAAGKGPEWVAV |
| | Heavy | ISYDGSNKLYADSVKGRFTISRDNSKNTLYLQMNSLRAEDTAVYYCARVP |
| | Chain | VGYYYYYGMDVWGQGTPVTVSSASTKGPSVFPLAPSSKSTSGGTAALGCL |
| | | VKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTP |
| | | PVLDSDGSFFLYSKLTVDKSRWQQGNVFSCSVMHEALHNHYTQKSLSLSP |
| | | GK |
| 32 | Full | EVQLLESGGGVVQPGRSLRLSCAASGFMFSNYWMIWVRQAAGKGPEWVAV |
| | Heavy | ISYDGSNKLYADSVKGRFTISRDNSKNTLYLQMNSLRAEDTAVYYCARVP |
| | Chain | VGYYYYYSMDVWGQGTPVTVSSASTKGPSVFPLAPSSKSTSGGTAALGCL |
| | | VKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTP |
| | | PVLDSDGSFFLYSKLTVDKSRWQQGNVFSCSVMHEALHNHYTQKSLSLSP |
| | | GK |
| 33 | Heavy | EVQLLESGGGVVQPGRSLRLSCAASGFMFSNYWMIWVRQAAGKGPEWVAV |
| | Full | ISYDGSNKLYADSVKGRFTISRDNSKNTLYLQMNSLRAEDTAVYYCARVP |
| | Chain | IAYYYYYGMDVWGQGTPVTVSSASTKGPSVFPLAPSSKSTSGGTAALGCL |
| | | VKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFPP |
| | | KPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTP |
| | | PVLDSDGSFFLYSKLTVDKSRWQQGNVFSCSVMHEALHNHYTQKSLSLSP |
| | | GK |

TABLE 14
Amino Acid sequences of full light
chains of a secondary anti-FRalpha antibody

| SEQ | | Sequence |
|---|---|---|
| ID | | 10 20 30 40 |
| NO. | Region | 12345678901234567890123456789012345678901234567890 |
| 34 | Full | AIRLTQSPSFLSASVGDRVTITCRASQGIRNYLAWYQQKPGKAPNLLIYA |
| | Light | ASLLQSGVPSRFSGSGFGTDFTLTISSLQPEDFATYYCQQASSFPLTFGG |
| | Chain | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |
| 35 | Full | AIRLTQSPSFLSASVGDRVTITCRASQGIRNYFAWYQQKPGKAPNLLIYA |
| | Light | ASLLQSGVPSRFSGSGFGTDFTLTISSLQPEDFATYYCQQASSFPFTFGG |
| | Chain | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |
| 36 | Full | AIRLTQSPSFLSASVGDRVTITCRASQGIRNYLAWYQQKPGKAPNLLIYA |
| | Light | ASLLQSGVPSRFSGSGFGTDFTLTISSLQPEDFATYYCQDASSFPLTFGG |
| | Chain | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |
| 37 | Full | AIRLTQSPSFLSASVGDRVTITCRASQGIRNYLAWYQQKPGKAPNLLIYA |
| | Light | ASLLQSGVPSRFSGSGFGTDFTLTISSLQPEDFATYYCQQASSFPVHFGG |
| | Chain | GTKVEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |

TABLE 15
Amino Acid sequences of variable heavy
chains of a secondary anti-FRalpha antibody

| SEQ | | Sequence |
|---|---|---|
| ID | | 10 20 30 40 |
| NO. | Region | 12345678901234567890123456789012345678901234567890 |
| 38 | VH | EVQLLESGGGVVQPGRSLRLSCAASGFMFSNYWMIWVRQAAGKGPEWVAV |
| | | ISYDGSNKLYADSVKGRFTISRDNSKNTLYLQMNSLRAEDTAVYYCARVP |
| | | VGYYYYYGMDVWGQGTPVTVSS |
| 39 | VH | EVQLLESGGGVVQPGRSLRLSCAASGFMFSNYWMIWVRQAAGKGPEWVAV |
| | | ISYDGSNKLYADSVKGRFTISRDNSKNTLYLQMNSLRAEDTAVYYCARVP |
| | | VGYYYYYSMDVWGQGTPVTVSS |
| 40 | VH | EVQLLESGGGVVQPGRSLRLSCAASGFMFSNYWMIWVRQAAGKGPEWVAV |
| | | ISYDGSNKLYADSVKGRFTISRDNSKNTLYLQMNSLRAEDTAVYYCARVP |
| | | IAYYYYYGMDVWGQGTPVTVSS |

TABLE 16
Amino Acid sequences of variable light
chains of a secondary anti-FRalpha antibody

| SEQ | | Sequence |
|---|---|---|
| ID | | 10 20 30 40 |
| NO. | Region | 12345678901234567890123456789012345678901234567890 |
| 41 | VL | AIRLTQSPSFLSASVGDRVTITCRASQGIRNYLAWYQQKPGKAPNLLIYA |
| | | ASLLQSGVPSRFSGSGFGTDFTLTISSLQPEDFATYYCQQASSFPLTFGG |
| | | GTKVEIK |
| 42 | VL | AIRLTQSPSFLSASVGDRVTITCRASQGIRNYFAWYQQKPGKAPNLLIYA |
| | | ASLLQSGVPSRFSGSGFGTDFTLTISSLQPEDFATYYCQQASSFPFTFGG |
| | | GTKVEIK |
| 43 | VL | AIRLTQSPSFLSASVGDRVTITCRASQGIRNYLAWYQQKPGKAPNLLIYA |
| | | ASLLQSGVPSRFSGSGFGTDFTLTISSLQPEDFATYYCQDASSFPLTFGG |
| | | GTKVEIK |
| 44 | VL | AIRLTQSPSFLSASVGDRVTITCRASQGIRNYLAWYQQKPGKAPNLLIYA |
| | | ASLLQSGVPSRFSGSGFGTDFTLTISSLQPEDFATYYCQQASSFPVHFGG |
| | | GTKVEIK |

Embodiments of the present disclosure are directed to methods and therapies comprising at least a pair of antibodies. In some aspects, the methods and therapies comprise a primary antibody and at least one other secondary antibody. According to embodiments of the present disclosure, the primary and any one or more secondary antibodies selected for a treatment are based at least in part on the epitopes to which of the antibodies bind. That is, the primary and any one or more secondary antibodies may be combined in a treatment based at least in part on the each of the antibodies binding to a non-overlapping epitope of the same target antigen.

The primary antibody may comprise a first variable domain comprising a nucleotide sequence that has at least 90 percent sequence identity or homology with SEQ ID NO: 1 (such as at least 95, 97, 98, or 99 percent sequence identity or homology), and the second variable domain comprising nucleotide sequence that has at least 90 percent sequence identity or homology with SEQ ID NO: 2 (such as at least 95, 97, 98, or 99 percent sequence identity or homology).

The primary antibody may comprise a first variable domain comprising an amino acid sequence that has at least 90 percent sequence identity or homology with SEQ ID NO: 3 (such as at least 95, 97, 98, or 99 percent sequence identity or homology), and the second variable domain comprising an amino acid sequence that has at least 90 percent sequence identity or homology with SEQ ID NO: 4 (such as at least 95, 97, 98, or 99 percent sequence identity or homology).

The secondary antibody may comprise a first variable domain comprising a nucleotide sequence that has at least 90 percent sequence identity or homology with SEQ ID NO: 5, 9, 13, 24, 25, or 26 (such as at least 95, 97, 98, or 99 percent sequence identity or homology), and the second variable domain comprising nucleotide sequence that has at least 90 percent sequence identity or homology with SEQ ID NO: 6, 10, 14, 27, 28, 29, or 30 (such as at least 95, 97, 98, or 99 percent sequence identity or homology).

The primary antibody may comprise a first variable domain comprising an amino acid sequence that has at least 90 percent sequence identity or homology with SEQ ID NO: 7, 11, 15, 38, 39, or 40 (such as at least 95, 97, 98, or 99 percent sequence identity or homology), and the second variable domain comprising an amino acid sequence that has at least 90 percent sequence identity or homology with SEQ ID NO: 8, 12, 16, 41, 42, 43, or 44 (such as at least 95, 97, 98, or 99 percent sequence identity or homology).

The secondary antibody may comprise a full heavy chain comprising a nucleotide sequence that has at least 90 percent sequence identity or homology with SEQ ID NO: 17, 18, or 19 (such as at least 95, 97, 98, or 99 percent sequence identity or homology), and a full light chain comprising nucleotide sequence that has at least 90 percent sequence identity or homology with SEQ ID NO: 20, 21, 22, or 23 (such as at least 95, 97, 98, or 99 percent sequence identity or homology).

The primary antibody may comprise a full heavy chain comprising an amino acid sequence that has at least 90 percent sequence identity or homology with SEQ ID NO: 31, 32, or 33 (such as at least 95, 97, 98, or 99 percent sequence identity or homology), and a full light chain comprising an amino acid sequence that has at least 90 percent sequence identity or homology with SEQ ID NO: 34, 35, 36, or 37 (such as at least 95, 97, 98, or 99 percent sequence identity or homology).

While it is presently described that SEQ ID NO: 1-4 are associated with a “primary” antibody and SEQ ID NO: 5-44 are associated with “secondary” antibodies, where a secondary antibody is paired with a primary antibody for a therapy, it should be understood that any of the antibodies disclosed herein can be used together in any combination. In some embodiments, methods and therapies of the present disclosure may combine multiple (e.g., one or more) secondary antibodies, with the primary antibody. In some embodiments, methods and therapies of the present disclosure may combine multiple secondary antibodies, without the primary antibody.

Having described various features of this disclosure both generally and specifically in the preceding detailed description, the following exemplification provides a specific example of the preparation of the subject matter described herein. By way of this example, and in the context of the preceding detailed description, the skilled person will immediately recognize variations to the method set forth in the example (such as by engineering a BiTE, scFv, or chimeric antigen receptor instead of an IgG). The following exemplification is illustrative only and shall not limit this disclosure or any patent claim that matures from this disclosure. Any patent claim that matures from this disclosure shall instead be limited by the explicit features recited in the claim in the context of its claim dependency and according to conventional principles of claim construction as applied in view of this disclosure.

### EXEMPLIFICATION

### Example 1. Development of Panel of Randomly Combined Human Genes Encoding Variable Regions that Bind FRalpha Extracellular Domain and Corresponding Fully-Human Anti-FRalpha IgG1 Antibodies

Antibodies disclosed herein were developed by the successful application of a extracellular domain of human FRalpha to a human v-gene phage display library. The library was constructed by randomly combining the variable genes encoding the immunoglobulin heavy chain (VH) with the variable genes encoding the immunoglobulin light chain (VL). The resulting library had a diversity of 10¹². Bio-panning of the library identified a panel of more than 35 positive colonies. Nucleotide sequencing of these colonies showed that they were all distinct (see Tables 1-8).

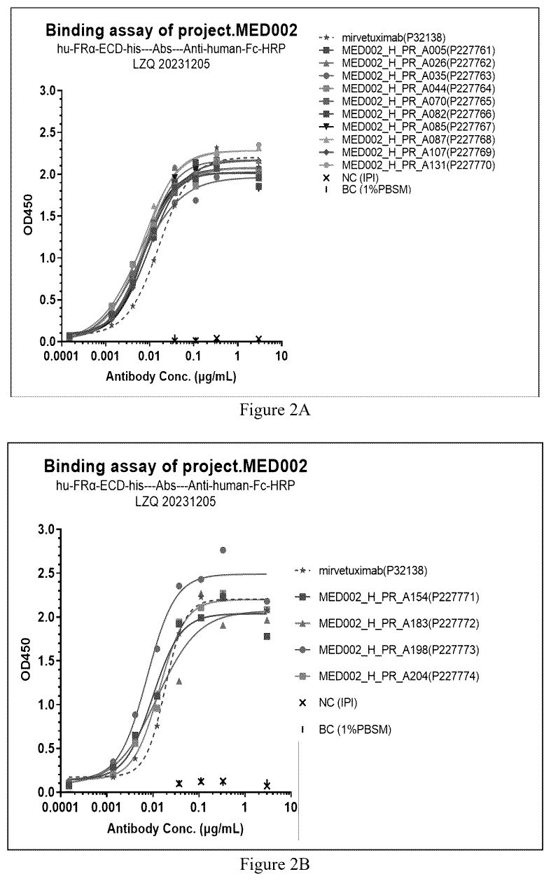

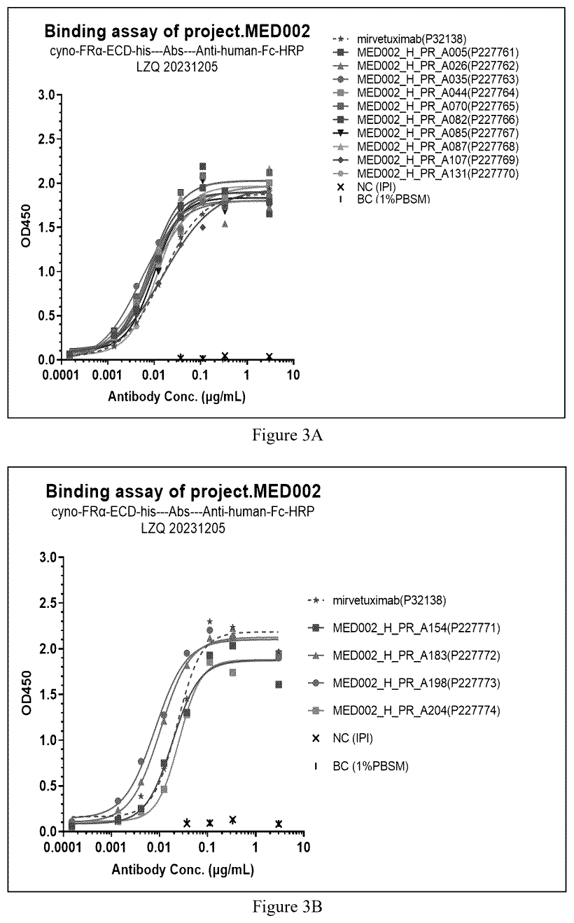

Of these, fourteen clones were randomly selected for further studies. Binding of the culture supernatant of cells engineered to transiently express the antibodies in ELISA plates coated with the human confirmed their binding ability (see FIGS. 2A and 2B, described in additional detail in Example 2). Since there is a very high degree of homology between human FRalpha and non-human primate FRalpha, it is not surprising that all the antibodies also bound to FRalpha from cynomolgus monkeys (see FIGS. 3A and 3B, described in additional detail in Example 3).

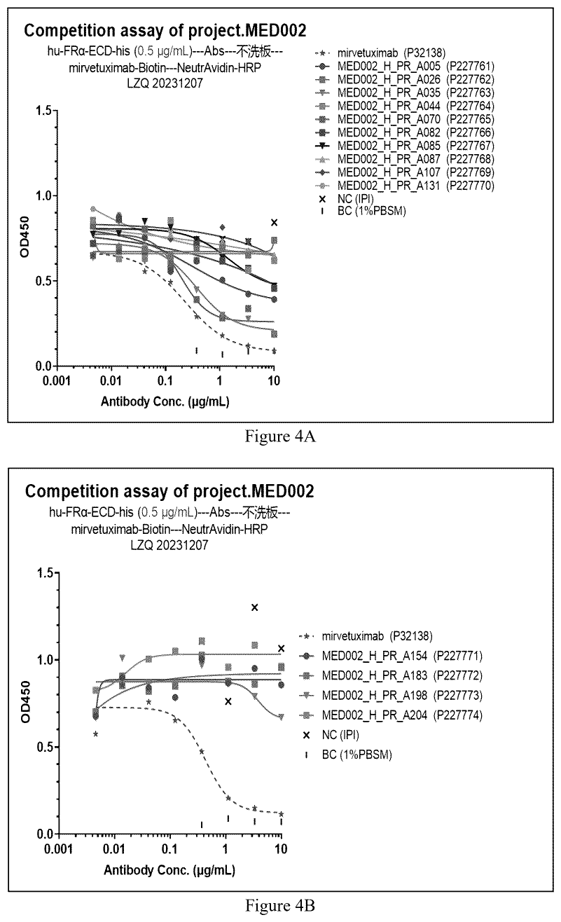

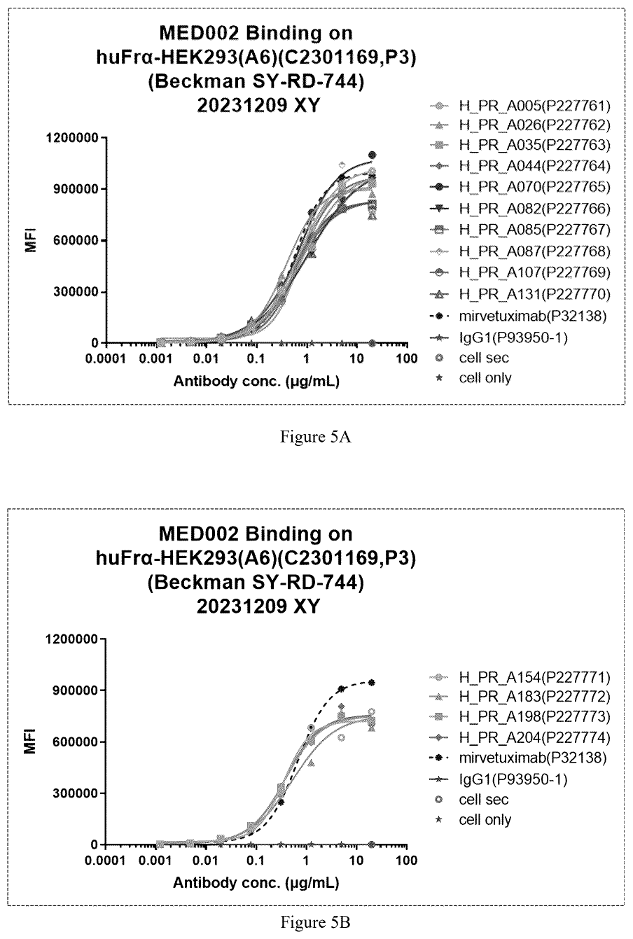

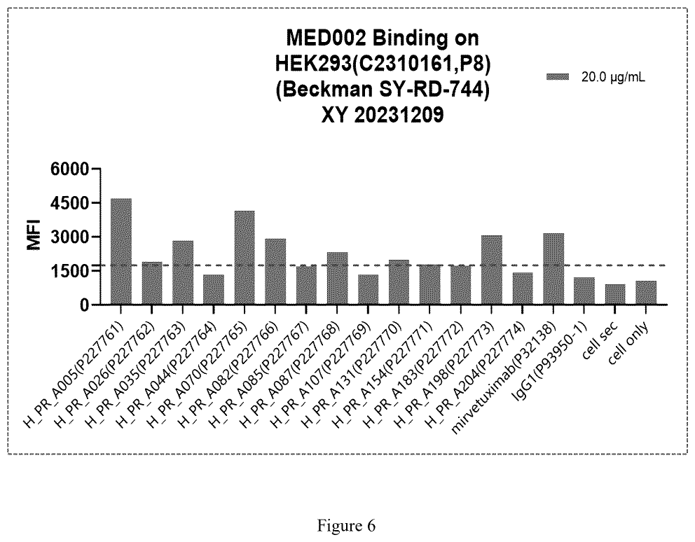

Interestingly, in competitive assays using ELISA, we found that the binding of four of these clones, MED002_H_PR_A0026 (P227762), MED002_H_PR_A044 (P227764), MED002_H_PR_A087 (P227768), and MED002_H_PR_A154 (P227771), did not block the binding of mirvetuximab while the binding of the other 10 clones were blocked, indicating that the antibodies produced by clones MED002_H_PR_A0026 (P227762), MED002_H_PR_A044 (P227764), MED002_H_PR_A087 (P227768), and MED002_H_PR_A154 (P227771) are directed at a non-overlapping B-cell epitope on human FRalpha (see FIGS. 4A and 4B, described in additional detail in Example 4). Antibodies produced by all four of these clones also bind to FRalpha expressed on HEK293 cells that have been engineered to over-express the human FRalpha (FIGS. 5A and 5B, described in additional detail in Example 5). However, only three of these clones, MED002_H_PR_A0026 (P227762), MED002_H_PR_A087 (P227768), and MED002_H_PR_A154 (P227771) bound significantly to the native HEK293 cells that express human FRalpha (FIG. 6, described in additional detail in Example 6).

The variable region nucleotide sequences and amino acid sequences of MED002_H_PR_A0026 (P227762) are provided in Tables 3 and 4, respectively. The variable region nucleotide sequences and amino acid sequences of MED002_H_PR_A087 (P227768) are provided in Tables 5 and 6, respectively. The variable region nucleotide sequences and amino acid sequences of MED002_H_PR_A154 (P227771) are provided in Tables 7 and 8, respectively.

Without being limited to any one theory, the results indicate the successful isolation of pairs of human FRalpha antibodies that bind to non-overlapping B-cell epitopes on the human FRalpha. According to embodiment of the present disclosure, such paired antibodies directed at two distinct and non-overlapping B-cell epitopes in an ADC or RIC formulation can be employed to functionally increase the copy number of a targeted antigen to enhance the efficacy of an antibody-based therapy.

### Example 2. Detecting Binding Activity of Fully-Human Anti-FRalpha IgG1 Antibodies to the Antigen, hu-FRα-ECD-his (P61020)

The affinity of each of the fourteen IgG1, referenced in Example 1, for the antigen, hu-FRα-ECD-his (P61020) was detected by ELISA. Briefly, a fusion protein containing the extracellular domain of human FRalpha and a polyhistidine tag was used to coat ELISA plates. Specifically, the ELISA plates were coated with 2 μg/mL hu-FRα-ECD-his (P61020) in 1×PBS at 30 μL per well and allowed to incubate overnight at approximately 4° C. After incubation, the plates were washed three times with polysorbate 20 (PBST), then blocked with 5 percent powdered milk (PBSM) for about 2 hours at approximately room temperature, and then washed three more times with PBST. Wells were then incubated with antibodies at varying concentrations for 60 minutes at room temperature in 1 percent PBSM. Wells were then washed three times with PBST, after which the secondary, horseradish-peroxidase-conjugated antibody anti-human-IgG-Fc-HRP (ab97225, Abcam, United Kingdom) was added at a 1:8000 dilution in 1 percent PBSM for about 50 minutes at room temperature. The wells were then washed six times with PBST, after which 3,3′,5,5″-tetramethylbenzidine (TMB) was added. The reaction was stopped with 2 molar sulfuric acid.

The optical density (OD) 450 (OD450) results of the fourteen antibodies at varying concentrations are depicted in FIGS. 2A and 2B. The half maximal effective concentration (EC50) results of the fourteen antibodies are provided in Tables 17 and 18. The antibody according to SEQ ID NO: 1-4 (MED002_H_PR_A070 (P227765)) displayed an EC₅₀ of 0.007149 micrograms per milliliter. The antibody according to SEQ ID NO: 5-8 (MED002_H_PR_A026 (P227762)) displayed an EC₅₀ of 0.007410 micrograms per milliliter. The antibody according to SEQ ID NO: 9-12 (MED002_H_PR_A087 (P227768)) displayed an EC₅₀ of 0.006509 micrograms per milliliter. The antibody according to SEQ ID NO: 13-16 (MED002_H_PR_A154 (P227771)) displayed an EC₅₀ of 0.009926 micrograms per milliliter. Mirvetuximab was assessed as a positive control and displayed an EC₅₀ of 0.01609 micrograms per milliliter for the run with the antibodies associated with SEQ ID NO: 5-12 and an EC₅₀ of 0.01853 micrograms per milliliter for the run with the antibody associated with SEQ ID NO: 13-16.

According to the results, each of the fourteen antibodies bound to hu-FRα-ECD-his (P61020).

TABLE 17
EC₅₀ values of fully-human anti-FRalpha IgG1 antibodies.

| Antibody | |
|---|---|
| Identifier | EC₅₀ |
| Mirvetuxim ab (P321138) | 0.01609 |
| MED002_H_PR_A005 (P227761) | 0.007410 |
| MED002_H_PR_A026 (P227762) | 0.007852 |
| MED002_H_PR_A035 (P227763) | 0.005973 |
| MED002_H_PR_A044 (P227764) | 0.005547 |
| MED002_H_PR_A070 (P227765) | 0.007149 |
| MED002_H_PR_A082 (P227766) | 0.007748 |
| MED002_H_PR_A085 (P227767) | 0.007309 |
| MED002_H_PR_A087 (P227768) | 0.006509 |
| MED002_H_PR_A107 (P227769) | 0.009704 |
| MED002_H_PR_A131 (P227770) | 0.007644 |

TABLE 18
EC₅₀ values of fully-human anti-FRalpha IgG1 antibodies.

| Antibody | |
|---|---|
| Identifier | EC₅₀ |
| Mirvetuxim ab (P321138) | 0.01853 |
| MED002_H_PR_A154 (P227771) | 0.009926 |
| MED002_H_PR_A183 (P227772) | 0.01231 |
| MED002_H_PR_A198 (P227773) | 0.007472 |
| MED002_H_PR_A204 (P227774) | 0.01371 |

### Example 3. Detecting Binding Activity of Fully-Human Anti-FRalpha IgG1 Antibodies to the Antigen, cyno-FRα-ECD-his (P61021)

The affinity of each of the fourteen IgG1, referenced in Example 1, for the antigen, cyno-FRα-ECD-his (P61021) was detected by ELISA. Briefly, a fusion protein containing the extracellular domain of cynomolgus monkey FRalpha and a polyhistidine tag was used to coat ELISA plates. Specifically, the ELISA plates were coated with 2 μg/mL cyno-FRα-ECD-his (P61021) in 1×PBS at 30 μL per well and allowed to incubate overnight at approximately 4° C. After incubation, the plates were washed three times with polysorbate 20 (PBST), then blocked with 5 percent powdered milk (PBSM) for about 2 hours at approximately room temperature, and then washed three more times with PBST. Wells were then incubated with antibodies at varying concentrations for 60 minutes at room temperature in 1 percent PBSM. Wells were then washed three times with PBST, after which the secondary, horseradish-peroxidase-conjugated antibody anti-human-IgG-Fc-HRP (ab97225, Abcam, United Kingdom) was added at a 1:8000 dilution in 1 percent PBSM for about 50 minutes at room temperature. The wells were then washed six times with PBST, after which 3,3′,5,5″-tetramethylbenzidine (TMB) was added. The reaction was stopped with 2 molar sulfuric acid.

The optical density (OD) 450 (OD450) results of the fourteen antibodies at varying concentrations are depicted in FIGS. 3A and 3B. The half maximal effective concentration (EC50) results of the fourteen antibodies are provided in Tables 19 and 20. The antibody according to SEQ ID NO: 1-4 (MED002_H_PR_A070 (P227765)) displayed an EC50 of 0.008028 micrograms per milliliter. The antibody according to SEQ ID NO: 5-8 (MED002_H_PR_A026 (P227762)) displayed an EC50 of 0.007772 micrograms per milliliter. The antibody according to SEQ ID NO: 9-12 (MED002_H_PR_A087 (P227768)) displayed an EC50 of 0.008048 micrograms per milliliter. The antibody according to SEQ ID NO: 13-16 (MED002_H_PR_A154 (P227771)) displayed an EC50 of 0.01856 micrograms per milliliter. Mirvetuximab was assessed as a positive control and displayed an EC50 of 0.0157 micrograms per milliliter for the run with the antibodies associated with SEQ ID NO: 5-12 and an EC50 of 0.02316 micrograms per milliliter for the run with the antibody associated with SEQ ID NO: 13-16.

According to the results, each of the fourteen antibodies bound to cyno-FRα-ECD-his (P61021). Since there is a very high degree of homology between human FRalpha and non-human primate FRalpha, it is not surprising that all the antibodies also bound to FRalpha from cynomolgus monkeys.

TABLE 19
EC₅₀ values of fully-human anti-FRalpha IgG1 antibodies.

| Antibody | |
|---|---|
| Identifier | EC₅₀ |
| Mirvetuxim ab (P321138) | 0.01517 |
| MED002_H_PR_A005 (P227761) | 0.009160 |
| MED002_H_PR_A026 (P227762) | 0.007772 |
| MED002_H_PR_A035 (P227763) | 0.005050 |
| MED002_H_PR_A044 (P227764) | 0.008833 |
| MED002_H_PR_A070 (P227765) | 0.008028 |
| MED002_H_PR_A082 (P227766) | 0.008208 |
| MED002_H_PR_A085 (P227767) | 0.009839 |
| MED002_H_PR_A087 (P227768) | 0.008048 |
| MED002_H_PR_A107 (P227769) | 0.01600 |
| MED002_H_PR_A131 (P227770) | 0.01241 |

TABLE 20
EC₅₀ values of fully-human anti-FRalpha IgG1 antibodies.

| Antibody | |
|---|---|
| Identifier | EC₅₀ |
| Mirvetuxim ab (P321138) | 0.02326 |
| MED002_H_PR_A154 (P227771) | 0.01856 |
| MED002_H_PR_A183 (P227772) | 0.01047 |
| MED002_H_PR_A198 (P227773) | 0.008114 |
| MED002_H_PR_A204 (P227774) | 0.02549 |

### Example 4. Detecting Anti-FRalpha IgG1 Antibody Competition Effect by ELISA

The competition of each of the fourteen IgG1, referenced in Example 1, for binding to FRα-ECD-his (P61020) was detected by ELISA. Briefly, a fusion protein containing the extracellular domain of human FRalpha and a polyhistidine tag was used to coat ELISA plates. Specifically, the ELISA plates were coated with 0.5 μg/mL hu-FRα-ECD-his (P61020) in 1×PBS at 30 μL per well and allowed to incubate overnight at approximately 4° C. After incubation, the plates were washed three times with polysorbate 20 (PBST), then blocked with 5 percent powdered milk (PBSM) for about 2 hours at approximately room temperature, and then washed three more times with PBST. Wells were then incubated with the antibodies at varying concentrations for 60 minutes at room temperature in 1 percent PBSM. Mirvetuximab-Biotin (P20405B5.56) was then added to the wells at 1 μg/mL in 1% PBSM at 30 μL per well and allowed to incubate for about 60 minutes at room temperature. Wells were then washed three times with PBST, after which a peroxidase-conjugated form of avidin biotin-binding protein, NeutrAvidin-HRP (Thermo Fisher; 31001), was added at a 1:2000 dilution in 1 percent PBSM for about 50 minutes at room temperature. The wells were then washed six times with PBST, after which 3,3′,5,5″-tetramethylbenzidine (TMB) was added. The reaction was stopped with 2 molar sulfuric acid.

The optical density (OD) 450 (OD450) results of the fourteen antibodies at varying concentrations are depicted in FIGS. 4A and 4B. The half maximal inhibitory concentration (IC50) results of the fourteen antibodies are provided in Tables 21 and 22. The antibody according to SEQ ID NO: 1-4 (MED002_H_PR_A070 (P227765)) displayed an IC50 of 0.1952 micrograms per milliliter. The antibody according to SEQ ID NO: 5-8 (MED002_H_PR_A026 (P227762)) displayed an IC50 of approximately 12.01 micrograms per milliliter. The antibody according to SEQ ID NO: 9-12 (MED002_H_PR_A087 (P227768)) displayed an IC50 of approximately 5.810e+20 micrograms per milliliter. The antibody according to SEQ ID NO: 13-16 (MED002_H_PR_A154 (P227771)) displayed an IC50 of approximately 0.003763 micrograms per milliliter. Mirvetuximab was assessed as a positive control and displayed an IC50 of 0.2306 micrograms per milliliter for the run with the antibodies associated with SEQ ID NO: 5-12 and an IC50 of 0.4327 micrograms per milliliter for the run with the antibody associated with SEQ ID NO: 13-16.

Interestingly, according to the results, the binding of antibodies MED002_H_PR_A0026 (P227762), MED002_H_PR_A044 (P227764), MED002_H_PR_A087 (P227768), and MED002_H_PR_A154 (P227771) did not block the binding of mirvetuximab, suggesting that they bound to a non-overlapping B cell epitope on the FRalpha. In contrast, the other antibodies bound to an overlapping B-cell epitope on the FRalpha.

TABLE 21
IC₅₀ values of fully-human anti-FRalpha IgG1 antibodies.

| Antibody | |
|---|---|
| Identifier | IC₅₀ |
| Mirvetuxim ab (P321138) | 0.2306 |
| MED002_H_PR_A005 (P227761) | 0.3081 |
| MED002_H_PR_A026 (P227762) | ~12.01 |
| MED002_H_PR_A035 (P227763) | 0.3653 |
| MED002_H_PR_A044 (P227764) | ~0.0006191 |
| MED002_H_PR_A070 (P227765) | 0.1952 |
| MED002_H_PR_A082 (P227766) | ~78113560 |
| MED002_H_PR_A085 (P227767) | 1.457 |
| MED002_H_PR_A087 (P227768) | ~5.810e+020 |
| MED002_H_PR_A107 (P227769) | ~221212667 |
| MED002_H_PR_A131 (P227770) | ~2.889e−013 |

TABLE 22
IC₅₀ values of fully-human anti-FRalpha IgG1 antibodies.

| Antibody | |
|---|---|
| Identifier | IC₅₀ |
| Mirvetuxim ab (P321138) | 0.4327 |
| MED002_H_PR_A154 (P227771) | ~0.003763 |
| MED002_H_PR_A183 (P227772) | ~6.149e−007 |
| MED002_H_PR_A198 (P227773) | 3.864 |
| MED002_H_PR_A204 (P227774) | 0.01566 |

### Example 5. Detecting Binding Activity of Fully-Human Anti-FRalpha IgG1 Antibodies to HEK293 Cells and FRalpha Expressed on the HEK293 Cells

The binding of each of the fourteen IgG1, referenced in Example 1, for binding to huFrα-HEK293 (A6) (C2301169, P1) cells, a HEK293 cell line engineered to overexpress FRalpha, was detected by flow cytometry. The cells were plated at 1E5 cell/well. The cells were then centrifuged and washed with fluorescence-activated cell sorting (FACS) buffer. The antibodies were then added to the wells at varying concentrations at 100 μL per well and allowed to incubate for 60 minutes at about 4° C. The wells were then centrifuged and washed twice with FACS buffer. 100 μL of phycoerythrin (PE) antibody labeled Anti-Human IgG, Fcγ (PE) (Jackson 109-115-098 1:200) was added into the cells and allowed to incubate at for 30 minutes at about 4° C. The cells were resuspended with FACS buffer, and tested by flow cytometry.

The mean fluorescence intensity (MFI) results of the fourteen antibodies at varying concentrations are depicted in FIGS. 5A and 5B, indicating binding of the antibodies to FRalpha expressed on HEK293 cells that have been engineered to over-express the human FRalpha. The half maximal effective concentration (EC50) results of the fourteen antibodies are provided in Tables 23 and 24. The antibody according to SEQ ID NO: 1-4 (MED002_H_PR_A070 (P227765)) displayed an EC50 of 0.6875 micrograms per milliliter. The antibody according to SEQ ID NO: 5-8 (MED002_H_PR_A026 (P227762)) displayed an EC50 of 0.3883 micrograms per milliliter. The antibody according to SEQ ID NO: 9-12 (MED002_H_PR_A087 (P227768)) displayed an EC50 of 0.6795 micrograms per milliliter. The antibody according to SEQ ID NO: 13-16 (MED002_H_PR_A154 (P227771)) displayed an EC50 of 0.3538 micrograms per milliliter. Mirvetuximab was assessed as a positive control and displayed an EC50 of 0.5639 micrograms per milliliter for the run with the antibodies associated with SEQ ID NO: 5-12 and an EC50 of 0.6724 micrograms per milliliter for the run with the antibody associated with SEQ ID NO: 13-16. According to the results depicted in FIGS. 5A and 5B, antibodies produced by each of the fourteen clones bound to the FRalpha expressed on the HEK293 cells. The antibody, MED002_H_PR_A070 (P227765), displayed better binding performance to the FRalpha than the positive antibody mirvetuximab (P32138).

The MFI results for of the fourteen antibodies at 20 μg/mL are depicted in FIG. 6 indicating binding of the antibodies to the native HEK293 cells that express human FRalpha. According to the results depicted in FIG. 6, clones MED002_H_PR_A044 (P227764), MED002_H_PR_A085 (P227767), MED002_H_PR_A107 (P227769), MED002_H_PR_A183 (P227772), MED002_H_PR_A204 (P227774) did not have binding activity with the HEK293 cells. While all other clones did bind to the HEK293 cells, only three of the clones, MED002_H_PR_A0026 (P227762), MED002_H_PR_A087 (P227768), and MED002_H_PR_A154 (P227771) bound significantly to the native HEK293 cells that express human FRalpha.

TABLE 23
EC₅₀ values of fully-human anti-FRalpha IgG1 antibodies.

| Antibody | EC₅₀ |
|---|---|
| Identifier | (μg/mL) |
| Mirvetuxim ab (P321138) | 0.5639 |
| MED002_H_PR_A005 (P227761) | 0.6975 |
| MED002_H_PR_A026 (P227762) | 0.3883 |
| MED002_H_PR_A035 (P227763) | 0.8322 |
| MED002_H_PR_A044 (P227764) | 0.737 |
| MED002_H_PR_A070 (P227765) | 0.6875 |
| MED002_H_PR_A082 (P227766) | 1.056 |
| MED002_H_PR_A085 (P227767) | 0.4713 |
| MED002_H_PR_A087 (P227768) | 0.6795 |
| MED002_H_PR_A107 (P227769) | 0.6066 |
| MED002_H_PR_A131 (P227770) | 0.5268 |

TABLE 24
EC₅₀ values of fully-human anti-FRalpha IgG1 antibodies.

| Antibody | EC₅₀ |
|---|---|
| Identifier | (μg/mL) |
| Mirvetuxim ab (P321138) | 0.6724 |
| MED002_H_PR_A154 (P227771) | 0.3538 |
| MED002_H_PR_A183 (P227772) | 0.5335 |
| MED002_H_PR_A198 (P227773) | 0.3761 |
| MED002_H_PR_A204 (P227774) | 0.3792 |

### Example 6. Detecting Inhibition and Affinity Characteristics of Fully-Human Anti-FRalpha IgG1 Antibodies Using Biolayer Interferometry (BLI)

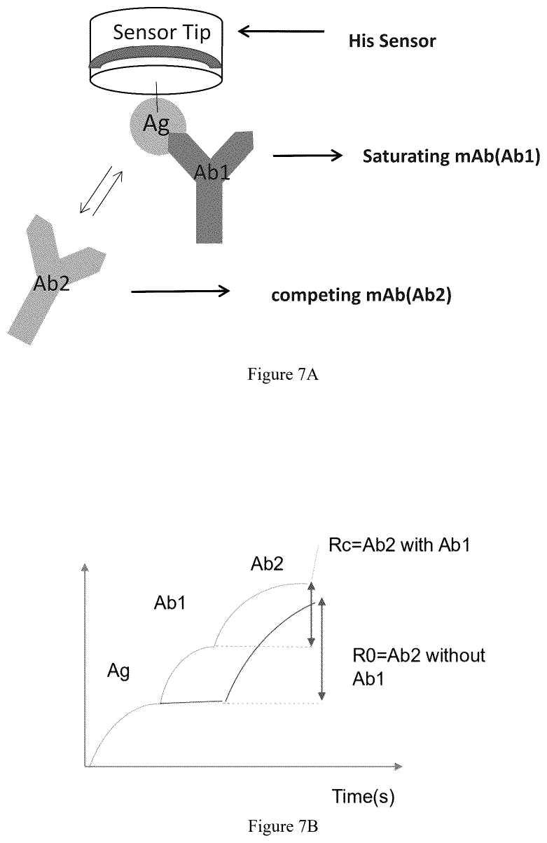

Biolayer Interferometry (BLI) is an optical technique for measuring macromolecular interactions by analyzing interference patterns of white light reflected from the surface of a biosensor tip. BLI experiments are used to determine the kinetics and affinity of molecular interactions. Specifically, in the context of this example and as depicted in FIG. 7A, a His-tagged antigen (i.e., Ag) is immobilized onto a biosensor tip (i.e., His sensor), which then allows the specific antibodies (i.e., primary or saturating mAb, Ab1, and secondary or competing mAb, Ab2) in a sample to bind to the antigen, causing a measurable shift in the interference pattern of light reflected from the sensor surface, thereby indicating the presence and binding affinity of the antibody to the antigen. FIG. 7B represents an example BLI graph, in which there are three noticeable phases in time. The first phase represents loading of the antigen, Ag, to the sensor, the second represents loading of the primary antibody, and the third represents loading of the secondary antibody. The inhibition rate is calculated as a percent by dividing Rc by R0, both depicted in FIG. 7B. If the inhibition rate for two antibodies is less than 20%, the two antibodies strongly compete. If the inhibition rate for two antibodies is between about 20% and 70% the two antibodies express intermediate competition. If the inhibition rate for two antibodies is greater than 70%, the two antibodies can be deemed as non-competing.

The BLI experiment was performed with a kinetics buffer (Q buffer) comprising 10 mM PBS with a pH of about 7.4, about 0.02% Tween 20 and about 0.2% BSA. Regeneration was performed with an DI water, 10 mM glycine, 150 mM sodium chloride at a pH of about 1.75 (R buffer). Example 6 was performed with a primary, saturation antibody A070 (referred to herein as MED002_H_PR_A070 (P227765) and associated with SEQUENCE ID NO. 1-4), and secondary, competing antibodies: MED019-008-007, MED019-008-033, MED019-008-051, MED019-008-086.

MED019-008-007 comprises a heavy chain sequence represented by SEQ ID NO: 18, 25, 32, and 39. MED019-008-007 comprises a light chain represented by SEQ ID NO: 21, 28, 35, and 42. That is, antibody MED019-008-007 comprises a heavy chain with a nucleotide sequence and an amino acid sequence with at least 70%, or more particularly at least 80%, or more particularly at least 90%, or more particularly at least 95%, or even more particularly 100% homology with SEQ ID NO: 18 and 32, respectively. The variable region of the MED019-008-007 heavy chain comprises a nucleotide sequence and an amino acid sequence with at least 70%, or more particularly at least 80%, or more particularly at least 90%, or more particularly at least 95%, or even more particularly 100% homology with SEQ ID NO: 25 and 39, respectively. MED019-008-007 comprises a light chain with a nucleotide sequence and an amino acid sequence with at least 70%, or more particularly at least 80%, or more particularly at least 90%, or more particularly at least 95%, or even more particularly 100% homology with SEQ ID NO: 21 and 35, respectively. The variable region of the MED019-008-007 light chain comprises a nucleotide sequence and an amino acid sequence with at least 70%, or more particularly at least 80%, or more particularly at least 90%, or more particularly at least 95%, or even more particularly 100% homology with SEQ ID NO: 28 and 42, respectively.

MED019-008-033 comprises a heavy chain sequence represented by SEQ ID NO: 17, 24, 31, and 38. MED019-008-033 comprises a light chain represented by SEQ ID NO: 22, 29, 36, and 43. That is, antibody MED019-008-033 comprises a heavy chain with a nucleotide sequence and an amino acid sequence with at least 70%, or more particularly at least 80%, or more particularly at least 90%, or more particularly at least 95%, or even more particularly 100% homology with SEQ ID NO: 17 and 31, respectively. The variable region of the MED019-008-033 heavy chain comprises a nucleotide sequence and an amino acid sequence with at least 70%, or more particularly at least 80%, or more particularly at least 90%, or more particularly at least 95%, or even more particularly 100% homology with SEQ ID NO: 24 and 38, respectively. MED019-008-033 comprises a light chain with a nucleotide sequence and an amino acid sequence with at least 70%, or more particularly at least 80%, or more particularly at least 90%, or more particularly at least 95%, or even more particularly 100% homology with SEQ ID NO: 22 and 36, respectively. The variable region of the MED019-008-033 light chain comprises a nucleotide sequence and an amino acid sequence with at least 70%, or more particularly at least 80%, or more particularly at least 90%, or more particularly at least 95%, or even more particularly 100% homology with SEQ ID NO: 29 and 43, respectively.

MED019-008-051 comprises a heavy chain sequence represented by SEQ ID NO: 19, 26, 33, and 40. MED019-008-051 comprises a light chain represented by SEQ ID NO: 20, 27, 34, and 41. That is, antibody MED019-008-051 comprises a heavy chain with a nucleotide sequence and an amino acid sequence with at least 70%, or more particularly at least 80%, or more particularly at least 90%, or more particularly at least 95%, or even more particularly 100% homology with SEQ ID NO: 19 and 33, respectively. The variable region of the MED019-008-051 heavy chain comprises a nucleotide sequence and an amino acid sequence with at least 70%, or more particularly at least 80%, or more particularly at least 90%, or more particularly at least 95%, or even more particularly 100% homology with SEQ ID NO: 26 and 40, respectively. MED019-008-051 comprises a light chain with a nucleotide sequence and an amino acid sequence with at least 70%, or more particularly at least 80%, or more particularly at least 90%, or more particularly at least 95%, or even more particularly 100% homology with SEQ ID NO: 20 and 34, respectively. The variable region of the MED019-008-051 light chain comprises a nucleotide sequence and an amino acid sequence with at least 70%, or more particularly at least 80%, or more particularly at least 90%, or more particularly at least 95%, or even more particularly 100% homology with SEQ ID NO: 27 and 41, respectively.

MED019-008-086 comprises a heavy chain sequence represented by SEQ ID NO: 17, 24, 31, and 38. MED019-008-086 comprises a light chain represented by SEQ ID NO: 23, 30, 37 and 44. That is, antibody MED019-008-086 comprises a heavy chain with a nucleotide sequence and an amino acid sequence with at least 70%, or more particularly at least 80%, or more particularly at least 90%, or more particularly at least 95%, or even more particularly 100% homology with SEQ ID NO: 17 and 31, respectively. The variable region of the MED019-008-086 heavy chain comprises a nucleotide sequence and an amino acid sequence with at least 70%, or more particularly at least 80%, or more particularly at least 90%, or more particularly at least 95%, or even more particularly 100% homology with SEQ ID NO: 24 and 38, respectively. MED019-008-086 comprises a light chain with a nucleotide sequence and an amino acid sequence with at least 70%, or more particularly at least 80%, or more particularly at least 90%, or more particularly at least 95%, or even more particularly 100% homology with SEQ ID NO: 23 and 37, respectively. The variable region of the MED019-008-086 light chain comprises a nucleotide sequence and an amino acid sequence with at least 70%, or more particularly at least 80%, or more particularly at least 90%, or more particularly at least 95%, or even more particularly 100% homology with SEQ ID NO: 30 and 44, respectively.

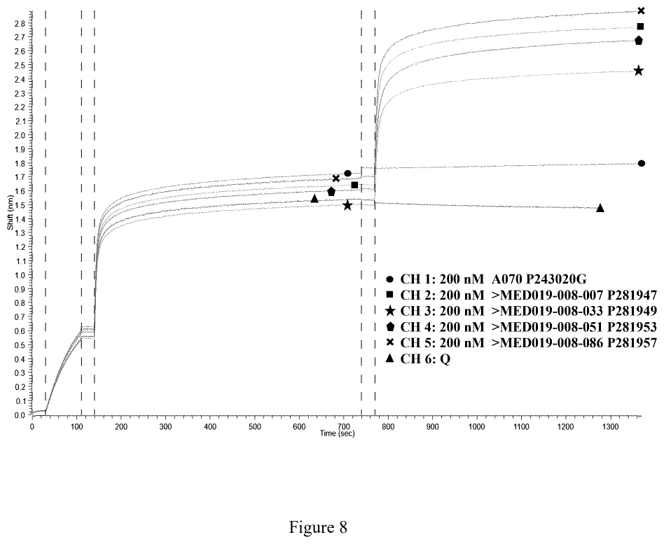

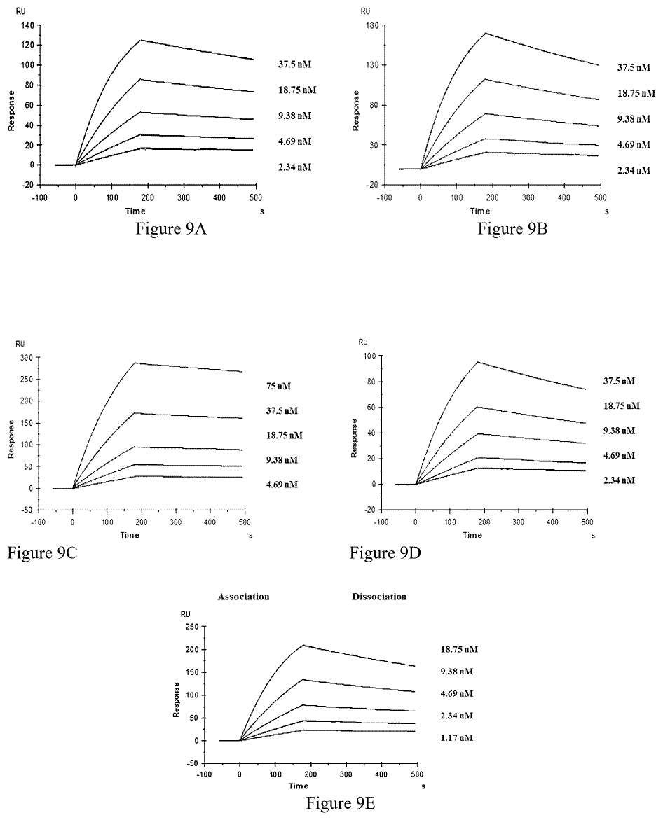

The inhibition results are provided in Table 25, and FIG. 8. According to the results, MED019-008-007, MED019-008-033, MED019-008-051, and MED019-008-086 antibodies each bind to an epitope that does not overlap with the epitope bound by A070 (the primary antibody). The affinity measurements by surface plasmon resonance (SPR) of the antibodies to FRalpha are provided in Table 26 and FIGS. 9A through 9E. FIG. 9A depicts the affinity of clone MED019-008-007 to FRalpha. FIG. 9B depicts the affinity of clone MED019-008-033 to FRalpha. FIG. 9C depicts the affinity of clone MED019-008-051 to FRalpha. FIG. 9D depicts the affinity of clone MED019-008-086 to FRalpha. FIG. 9E depicts the affinity of a control antibody to FRalpha. According to the results, the MED019-008-007, MED019-008-033, MED019-008-051, and MED019-008-086 antibodies each showed high affinity binding to FRalpha, with the KD in the region of nM.

TABLE 25
Inhibition rates of secondary antibodies to
a primary antibody binding the same antigen.

| | Ab2 | | | | | |
|---|---|---|---|---|---|---|
| | | Inhibition Rate (%) | | | | |
| | | | MED019- | MED019- | MED019- | MED019- |
| | | A070 | 008-007 | 008-033 | 008-051 | 008-086 |
| Ab1 | Number | P243020G | P281947 | P281949 | P281953 | P281957 |
| A070 | P243020G | 7.45% | 103.45% | 103.64% | 102.44% | 104.19% |

TABLE 26
Affinity Measurements of fully-human anti-
FRalpha IgG1 antibodies to hu-FRα-ECD-his.

| | | hu-FRα-ECD-his | | | | |
|---|---|---|---|---|---|---|
| Protein | Protein | KD | ka | kd | Rmax | |
| Name | No. | (M) | (1/Ms) | (1/s) | (RU) | Chi² |
| A070 | P243020G | 1.38E−09 | 6.96E+05 | 9.59E−04 | 267.3 | 0.296 |
| >MED019-008-007 | P281947 | 1.93E−09 | 2.96E+05 | 5.71E−04 | 157.0 | 0.123 |
| >MED019-008-033 | P281949 | 3.67E−09 | 2.40E+05 | 8.82E−04 | 230.4 | 0.119 |
| >MED019-008-051 | P281953 | 2.68E−09 | 8.16E+04 | 2.19E−04 | 437.8 | 0.363 |
| >MED019-008-086 | P281957 | 3.93E−09 | 2.33E+05 | 9.15E−04 | 137.8 | 0.0678 |

Without being limited to any one theory, the results herein indicate the successful isolation of pairs of human FRalpha antibodies that bind to non-overlapping B-cell epitopes on the human FRalpha. This enables the use of such paired antibodies directed at two distinct and non-overlapping B-cell epitopes in an ADC or RIC formulation to functionally increase the copy number of a targeted antigen to enhance the efficacy of an antibody-based therapy.

Without limitation, in some embodiments such formulations may provide at least one or more of the following potential advantages: (1) Functionally increase the copy number of the antigen to increase the applicability of the antibody-based therapy, potentially enabling its use with patients whose tumor cells express lower antigen copy numbers, who would otherwise not benefit from such therapies; (2) Increase the total number of antibody molecules bound to the tumor cells to increase efficacy of antitumor activities of the antibodies to facilitate ADCC and CDC; (3) As this approach involves two complementary antibodies, in some variants, use of such formulations could allow a medical professional to prepare and use formulations of combination chemotherapy, conjugating one antibody to one payload, and another antibody to another payload. In some instances the antibodies could mediate cytotoxicity using a different mechanism of action, thereby increasing the efficacy of the ADCs and reducing the risk of the development of chemoresistance. In contrast, a biparatopic bispecific ADC targeting two non-overlapping B-cell epitopes and would be unable to provide combination chemotherapy. Formulations of the present disclosure will also not be limited by whether or not the distance between the two epitopes on the antigen might be too far to accommodate the two binding sites on the bispecific antibody; and (4) In some embodiments, the formulations of the present disclosure will also ensure that if two different and synergistic payloads are being used, that all tumor cells will always be exposed to the same ratio of the two payloads.

In one aspect, a pair of FRalpha v-genes or/and their gene products may be used in the therapeutic development of, without limitation, naked antibodies, bispecific antibodies, BiTEs, ADC, RIC, and cell-based therapies such as CAR-T, CAR-NK, and CAR-monocytes.

Embodiments of the present disclosure are directed to the production of formulations of ADCs comprising two antibodies targeting two non-overlapping B-cell epitopes on FRalpha. In other aspects, formulations of ADCs comprising at least two antibodies targeting at least two non-overlapping B-cell epitopes on FRalpha are provided.

In still another aspect, the present disclosure provides formulations of combination chemotherapy ADCs that include at least two antibodies, each carrying a synergistic payload, targeting at least two non-overlapping B-cell epitopes. In others, the invention provides formulations of combination chemotherapy ADCs that include at least two antibodies, each carrying a synergistic payload, targeting at least two non-overlapping B-cell epitopes. Payloads may be varied to include synergistic compounds known to those of ordinary skill in the art.

Embodiments of the present disclosure are further directed to methods of increasing the exposure of chemotherapy agents in ADCs using formulations comprising at least two antibodies targeting at least two non-overlapping B-cell epitopes. In other embodiments, such methods utilize formulations comprising at least two antibodies targeting at least two non-overlapping B-cell epitopes.

In embodiments of the present disclose, methods of increasing the functional antigen density of tumor cells for therapies such as ADC therapy comprise using formulations comprising at least two antibodies targeting at least two non-overlapping B-cell epitopes. As above, such methods may also comprise using at least two antibodies targeting at least two non-overlapping B-cell epitopes.

Aspects of the present disclosure are therefore directed to novel therapies that are based on increasing the number of antibodies/ADC molecules that are able to attach to a target cell with a fixed number of antigens by functionally increasing the FRalpha antigen copy number. Therapies of the present disclosure enhance the intrinsic antitumor properties of the backbone antibodies (i.e., the naked antibody, without a payload) by ADCC, ADCP, and CDC. Additionally, the number of ADC molecules with payload delivered to each tumor cell is increased by functionally increasing the copy number of the FRalpha antigen. Therefore, the therapies disclosed herein may be effective even for patients whose tumor cells express a low copy number of the FRalpha antigen. Further, embodiment of the present disclosure utilize antibodies of 100% human sequence which reduces the likelihood for the development of anti-drug antibodies.

No patent claim that matures from this disclosure shall be interpreted as requiring any feature of the foregoing Example. Any methods described in the claims or specification shall not be interpreted to require the steps to be performed in a specific order unless expressly stated otherwise. The methods shall be interpreted to provide support to perform the recited steps in any order unless expressly stated otherwise.

Certain features described in the context of separate implementations can also be implemented in combination in a single implementation. Conversely, various features that are described in the context of a single implementation can also be implemented in multiple implementations separately or in any suitable sub-combination. Moreover, although features may be described above in certain combinations and even initially claimed as such, one or more features from a claimed combination can be excised from the combination, and the claimed combination may be directed to a sub-combination or variation of a sub-combination.

The example configurations described in this document do not represent all the examples that may be implemented or that fall within the scope of the claims. The term “example” shall be interpreted to mean “serving as an example, instance, or illustration,” and not “preferred” or “advantageous over other examples.”

Articles such as “the,” “a,” and “an” can connote the singular or plural. The word “or” when used without a preceding “either” (or other similar language indicating that “or” is unequivocally meant to be exclusive, for example, only one of x or y) shall be interpreted to be inclusive (for example, “x or y” means one or both of x and y).

The term “and/or” shall also be interpreted to be inclusive (for example, “x and/or y” means one or both of x and y). In situations where “and/or” or “or” are used as a conjunction for a group of three or more items, then the group shall be interpreted to include one item alone, all the items together, or any combination or number of the items.

The terms “has,” “contain(s),” and “include(s)” shall be interpreted to be synonymous with the term “comprise(s)” and as inclusive or open-ended such as to not exclude additional unrecited subject matter. Use of the four preceding terms also discloses and provides support for narrower alternative implementations, in which these terms are replaced by “consisting” or “consisting essentially of,” which are closed as to exclude additional unrecited subject matter.

Unless otherwise indicated, all numbers or expressions, such as those expressing concentrations, ratios, counts, and the like, used in the specification (other than the claims) are understood to be modified in all instances by the term “approximately.” At the very least, and not as an attempt to limit the application of the doctrine of equivalents to the claims, each numerical parameter recited in the specification or claims that is modified by the term “approximately” should be construed in light of the number of recited significant digits and by applying ordinary rounding techniques. All disclosed ranges are to be understood to encompass and provide support for claims that recite any subranges or any and all individual values subsumed by each range. For example, a stated range of “at least 90 percent” shall be construed as including support for at least 90 percent, at least 95 percent, at least 97 percent, at least 98 percent, at least 99 percent, at least 99.5 percent, at least 99.6 percent, at least 99.7 percent, at least 99.8 percent, and at least 99.9 percent.

The terms recited in the claims should be given their ordinary and customary meaning as determined by reference to relevant entries in widely used general dictionaries, relevant technical references, commonly understood meanings by those in the art, and the like with the understanding that the broadest meaning imparted by any one or combination of these sources should be given to the claim terms (for example, two or more relevant references should be combined to provide the broadest meaning of the combination of references) subject only to the following two exceptions: (a) when a term is used in a manner that is more expansive than its ordinary and customary meaning, then the term should be given its ordinary and customary meaning plus the additional expansive meaning, and (b) when a term has been explicitly defined to have a different meaning by reciting the term and its definition along with the phrase “in this disclosure” or similar language, then the term shall be limited to the definition. References to specific examples shall not invoke the foregoing exception (b) or otherwise restrict the scope of the recited claim terms. Other than situations where the foregoing exception (b) applies, nothing contained in this document should be considered a disclaimer or disavowal of claim scope.

The subject matter recited in the claims is not coextensive with and should not be interpreted to be coextensive with any implementation, feature, or combination of features described or illustrated in this document. This is true even if only a single implementation of the feature or combination of features is illustrated and described.

1. A method to functionally increase the copy number of a target antigen of a human subject, comprising:
administering, to the human subject, a set of immunotherapeutic agents that target an antigen, wherein the set of immunotherapeutic agents comprises:
a first immunotherapeutic agent comprising a first antibody conjugated with a first payload;
a least one second immunotherapeutic agent comprising a second antibody conjugated with a second payload,
wherein the first antibody and the second antibody are different and bind different, non-overlapping epitopes of the antigen, and
wherein the first payload and the second payload are the same or different.

2. The method of claim 1, wherein the set of immunotherapeutic agents consists of the first immunotherapeutic agent and a second immunotherapeutic agent, the first immunotherapeutic agent targeting a first epitope of the antigen and the second immunotherapeutic agent targeting a second epitope of the antigen, wherein the first epitope and the second epitope are distinct and non-overlapping.

3. The method of claim 1, wherein the first payload, the second payload, or both comprises a radioactive isotope selected from the group comprising actinium-225, astatine-211, bismuth-212, bismuth-213, copper-67, gallium-68, holmium-166, iodine-124, iodine-131, lutetium-177, samarium-153, technetium-99, terbium-149, and yttrium-90.

4. The method of claim 1, wherein the first payload, the second payload, or both comprises a pharmaceutical agent comprising a moiety selected from the group comprising amanitin, 3-aminophenyl hemiasterlin, calicheamicin, camptothecin, deruxtecan, doxorubicin, emtansine, eribulin, exatecan, irinotecan, maleimidocaproyl monomethyl auristatin F, maytansine, mertansine (N2′-deacetyl-N2′-(3-mercapto-1-oxopropyl)-maytansine; DM1), monomethyl auristatin F, paclitaxel, PE38, pyrrolobenzodiazepine, ravtansine (N2′-deacetyl-N2′-(4-mercapto-4-methyl-1-oxopentyl) maytansine; DM4), SN-38, and vedotin.

5. The method of claim 1, wherein the antigen is folate receptor alpha.

6. The method of claim 1, wherein the set of immunotherapeutic agents are administered to target an antigen and kill a cell associated with the target antigen via antibody-dependent cell-mediated cytotoxicity (ADCC), antibody-dependent cellular phagocytosis (ADCP), or complement-dependent cytotoxicity (CDC).

7. The method of claim 1, wherein the set of immunotherapeutic agents are administered via in an antibody-drug conjugates (ADC) or radioimmunoconjugates (RIC) formulation.

8. The method of claim 1, wherein the epitopes are b-cell epitopes.

9. The method of claim 1, wherein the first payload and the second payload are the same.

10. The method of claim 1, wherein the first payload and the second payload are different.

11. The method of claim 1, wherein the immunotherapeutic agent is an IgG antibody.

12. The method of claim 1, wherein:
the immunotherapeutic agent is administered at an effective amount that is effective to induce cell death in at least a portion of the cells that express folate receptor alpha; and
the method modulates the cells that express folate receptor alpha by inducing cell death.

13. A method of treating or preventing cancer in a human subject, comprising performing the method of claim 1, wherein:
the human subject presents with cancer; and
at least a portion of the cells that express folate receptor alpha are cancer cells.

14. The method of claim 1, comprising identifying that the human subject comprises cells that overexpress folate receptor alpha.

15. The method of claim 14, wherein the cells that overexpress folate receptor alpha comprise epithelial cells, fallopian tube cells, leukemia cells, epithelial cells, or a combination thereof.

16. The method of claim 1, comprising identifying that a tissue sample of the human subject comprises either RNA encoding folate receptor alpha or folate receptor alpha protein prior to the administering.

17. The method of claim 16, wherein the tissue sample is a blood sample.

18. The method of claim 16, wherein the tissue sample is a biopsy.

19. The method of claim 1, wherein the administering is selected from intravenous, intramuscular, subcutaneous, intradermal, intraocular, parenteral, intraperitoneal, intrathecal, intralesional, and intratumoral administering.

20. A set of immunotherapeutic agents for functionally increasing the copy number of a target antigen of a human subject, comprising:
a first immunotherapeutic agent comprising a first antibody conjugated with a first payload;
a least one second immunotherapeutic agent comprising a second antibody conjugated with a second payload,
wherein the first antibody and the second antibody are different and bind different, non-overlapping epitopes of the antigen, and
wherein the first payload and the second payload are the same or different.


SEQUENCE LISTING (44 sequences)

SEQ ID NO: 1 - DNA, 354 bp, Synthetic construct
  source 1..354: mol_type = other DNA
gaggtgcagctgttggagtctgggggaggcttggtcaagcctggggggtccctgagactctcctgtgcagcctctggattcaccttcagtagctataccatgaactgggtccgccaggctccagggaaggggctggaatgggtctcatccattagtagtggtggtggttacatatactacgcagactcagtgaaggaccgattcaccatctccagagacaacgccaagaactcactgtatctgcaaatgaacagcctgcgagccgaggacacggccgtgtattactgtgcaaaaggggatacgatcatgataatatcggcgtggggccaagggacaacggtcaccgtctcatca

SEQ ID NO: 2 - DNA, 321 bp, Synthetic construct
  source 1..321: mol_type = other DNA
gacatccagttgacccagtctccttccaccctgtctgcatctgtaggagacagagtcaccatcacttgccgggccagtcagagtattagtagttggttggcctggcatcagcagaaaccagggaaagcccctaaactcctcatctatgctgcatccagtttgcaaaatggggtcccatcaaggttcagcggcagtggatttgggacagatttcactctcaccatcagcagcctgcagcctgaagactttgcaacttactattgtcaacaggctagcagtttccctctcactttcggcggagggaccaaggtggagatcaaa

SEQ ID NO: 3 - protein, 118 aa, Synthetic construct
EVQLLESGGGLVKPGGSLRLSCAASGFTFSSYTMNWVRQAPGKGLEWVSSISSGGGYIYYADSVKDRFTISRDNAKNSLYLQMNSLRAEDTAVYYCAKGDTIMIISAWGQGTTVTVSS

SEQ ID NO: 4 - protein, 107 aa, Synthetic construct
DIQLTQSPSTLSASVGDRVTITCRASQSISSWLAWHQQKPGKAPKLLIYAASSLQNGVPSRFSGSGFGTDFTLTISSLQPEDFATYYCQQASSFPLTFGGGTKVEIK

SEQ ID NO: 5 - DNA, 357 bp, Synthetic construct
  source 1..357: mol_type = other DNA
gaggtgcagctgttggactctgggggaggcttggtccagcctgggaggtccctgagactctcctgtgcagcgtctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagaactgaagacacggccgtgtattactgtgcgagacaaagtgggagcttcgggtactttgactactggggccaaggaacaccggtcaccgtctcatca

SEQ ID NO: 6 - DNA, 321 bp, Synthetic construct
  source 1..321: mol_type = other DNA
gacatccagatgacccagtctccatcctccctgtctgcatctgtaggagacagagtcaccatcacttgccgggccagtcagaatatttttacctggttagcctggtatcagcagaaaccagggaaagcccctaaactcctgatttatggtgcatccactttgcagagtggggtcccatcaaggttcagcggcagcggatctgggacagacttcactctcaccatcagcagtctgcagcctgaagattttgcgacttactattgtcaacaggctaagagtttccctctcactttcggcggagggaccaaggtggagatcaaa

SEQ ID NO: 7 - protein, 119 aa, Synthetic construct
EVQLLDSGGGLVQPGRSLRLSCAASGFTFSSYSMNWVRQAPGKGLEWVSSISSSSSYIYYADSVKGRFTISRDNAKNSLYLQMNSLRTEDTAVYYCARQSGSFGYFDYWGQGTPVTVSS

SEQ ID NO: 8 - protein, 107 aa, Synthetic construct
DIQMTQSPSSLSASVGDRVTITCRASQNIFTWLAWYQQKPGKAPKLLIYGASTLQSGVPSRFSGSGSGTDFTLTISSLQPEDFATYYCQQAKSFPLTFGGGTKVEIK

SEQ ID NO: 9 - DNA, 375 bp, Synthetic construct
  source 1..375: mol_type = other DNA
gaggtgcagctgttggagtctgggggaggcgtggtccagcctgggaggtccctgagactctcctgtgcagcctctggattcaccttcagtagctatggcatgcactgggtccgccaggctccaggcaaggggctggagtgggtggccaacataaaggaagatggaagtgagaaatactatgtggactctgtgaagggccggttcaccatctccagagacaacgccaagaactcactggatctgcagatgaacagcctgagagccgaggacacggctgtgtattactgtgcaagaggggaacgcgatttttggagtggttcttactactacggtatggacgtctggggccaggggaccatggtcaccgtctcatca

SEQ ID NO: 10 - DNA, 321 bp, Synthetic construct
  source 1..321: mol_type = other DNA
aacatccagatgacccagtctccatcttccctgtctgcatctgtaggagacacagtcaccatcacttgtcgggcgagtcaggatgttagcaggtggttaggctggtatcagcagaaaccaggcaaagcccctcggctcctgatctctgctacatcccgtttgcaaagtggtgtcccatcaaggttcagcggcagtggatctgggacagaattcactctcatcatcagcaacctgcagccggaagattttgcgacttactactgtcaacaggtcaacaacttcccgatcaccttcggcggagggaccaaggtggagatcaag

SEQ ID NO: 11 - protein, 125 aa, Synthetic construct
EVQLLESGGGVVQPGRSLRLSCAASGFTFSSYGMHWVRQAPGKGLEWVANIKEDGSEKYYVDSVKGRFTISRDNAKNSLDLQMNSLRAEDTAVYYCARGERDFWSGSYYYGMDVWGQGTMVTVSS

SEQ ID NO: 12 - protein, 107 aa, Synthetic construct
NIQMTQSPSSLSASVGDTVTITCRASQDVSRWLGWYQQKPGKAPRLLISATSRLQSGVPSRFSGSGSGTEFTLIISNLQPEDFATYYCQQVNNFPITFGGGTKVEIK

SEQ ID NO: 13 - DNA, 354 bp, Synthetic construct
  source 1..354: mol_type = other DNA
caggtgcagctgttggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagcagcaacaactggtacggttgactactggggccagggaacaacggtcaccgtctcatca

SEQ ID NO: 14 - DNA, 321 bp, Synthetic construct
  source 1..321: mol_type = other DNA
gacatccagatgacccagtctccatcttccgtgtctgcatctgtgggagacagagtcaccattacttgtcgggcgagtcagggcattagcacctggttagcctggtatcagcagaaaccagggaaagcccctaaactcctcatctatgctgcatccagtttgcaaaatggggtcccatcaaggttcagcggcagtggatttgggacagatttcactctcaccatcagcagcctgcagcctgaagactttgcaacttactattgtcaacaggctagcagtttccctctcactttcggcggagggaccaaggtggagatcaaa

SEQ ID NO: 15 - protein, 118 aa, Synthetic construct
QVQLLESGGGLVKPGGSLRLSCAASGFTFSSYSMNWVRQAPGKGLEWVSSISSSSSYIYYADSVKGRFTISRDNAKNSLYLQMNSLRAEDTAVYYCARAATTGTVDYWGQGTTVTVSS

SEQ ID NO: 16 - protein, 107 aa, Synthetic construct
DIQMTQSPSSVSASVGDRVTITCRASQGISTWLAWYQQKPGKAPKLLIYAASSLQNGVPSRFSGSGFGTDFTLTISSLQPEDFATYYCQQASSFPLTFGGGTKVEIK

SEQ ID NO: 17 - DNA, 1356 bp, Synthetic construct
  source 1..1356: mol_type = other DNA
gaggtgcagctgttggagtctgggggaggcgtggtccagcctgggaggtccctgagactctcctgtgcagcctctggattcatgtttagtaactattggatgatttgggtccgtcaggctgcagggaagggtcctgagtgggtggcagttatatcatatgatggaagcaataaattgtacgcagactccgtgaagggccgattcaccatctccagagacaattccaagaacacgctgtatctgcaaatgaacagcctgagagctgaggacactgccgtgtattactgtgcgagagtaccagtaggttactactactactacggtatggacgtctggggtcaggggactccggtcaccgtctcatcagcttccaccaagggcccctccgtgttccccctggctccctcttccaagagcaccagcggcggcaccgctgctctgggatgtctggtgaaggactacttccctgagcctgtgaccgtgtcctggaattccggcgccctgacctccggcgtgcacacattccctgctgtgctgcagtcctccggcctgtatagcctgtcctccgtggtgacagtgcctagctccagcctgggcacccagacctatatctgcaacgtgaaccacaagcctagcaataccaaggtggacaagaaggtggagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaaa

SEQ ID NO: 18 - DNA, 1356 bp, Synthetic construct
  source 1..1356: mol_type = other DNA
gaggtgcagctgttggagtctgggggaggcgtggtccagcctgggaggtccctgagactctcctgtgcagcctctggattcatgtttagtaactattggatgatttgggtccgtcaggctgcagggaagggtcctgagtgggtggcagttatatcatatgatggaagcaataaattgtacgcagactccgtgaagggccgattcaccatctccagagacaattccaagaacacgctgtatctgcaaatgaacagcctgagagctgaggacactgccgtgtattactgtgcgagagtaccagtaggttactactactactactcgatggacgtctggggtcaggggactccggtcaccgtctcatcagcttccaccaagggcccctccgtgttccccctggctccctcttccaagagcaccagcggcggcaccgctgctctgggatgtctggtgaaggactacttccctgagcctgtgaccgtgtcctggaattccggcgccctgacctccggcgtgcacacattccctgctgtgctgcagtcctccggcctgtatagcctgtcctccgtggtgacagtgcctagctccagcctgggcacccagacctatatctgcaacgtgaaccacaagcctagcaataccaaggtggacaagaaggtggagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaaa

SEQ ID NO: 19 - DNA, 1356 bp, Synthetic construct
  source 1..1356: mol_type = other DNA
gaggtgcagctgttggagtctgggggaggcgtggtccagcctgggaggtccctgagactctcctgtgcagcctctggattcatgtttagtaactattggatgatttgggtccgtcaggctgcagggaagggtcctgagtgggtggcagttatatcatatgatggaagcaataaattgtacgcagactccgtgaagggccgattcaccatctccagagacaattccaagaacacgctgtatctgcaaatgaacagcctgagagctgaggacactgccgtgtattactgtgcgagagtaccaattgcttactactactactacggtatggacgtctggggtcaggggactccggtcaccgtctcatcagcttccaccaagggcccctccgtgttccccctggctccctcttccaagagcaccagcggcggcaccgctgctctgggatgtctggtgaaggactacttccctgagcctgtgaccgtgtcctggaattccggcgccctgacctccggcgtgcacacattccctgctgtgctgcagtcctccggcctgtatagcctgtcctccgtggtgacagtgcctagctccagcctgggcacccagacctatatctgcaacgtgaaccacaagcctagcaataccaaggtggacaagaaggtggagcctaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaaa

SEQ ID NO: 20 - DNA, 642 bp, Synthetic construct
  source 1..642: mol_type = other DNA
gccatccggttgacccagtctccatccttcctgtctgcatctgtaggagacagagtcaccatcacttgtcgtgccagtcagggcattaggaattatttagcctggtatcagcaaaaaccagggaaagctcctaacctcctgatctatgctgcatccttgttgcaaagtggggtcccatcaaggttcagcggcagtggatttgggacagatttcactctcaccatcagcagcctgcagcctgaagactttgcaacttactattgtcaacaggctagcagtttccctctcactttcggtggagggaccaaggtggagatcaaaaggaccgtggctgcccccagcgtgttcatcttccctcctagcgacgagcagctgaagagcggcaccgctagcgtggtgtgtctgctgaataacttctatcccagggaggccaaggtgcagtggaaggtggataacgccctgcagagcggcaactcccaggagtccgtgaccgagcaggactccaaggacagcacctactccctgagctccaccctgaccctgtccaaggctgattatgagaagcacaaggtgtatgcttgcgaggtgacacaccagggcctgtccagccctgtgaccaagagcttcaaccggggcgagtgc

SEQ ID NO: 21 - DNA, 642 bp, Synthetic construct
  source 1..642: mol_type = other DNA
gccatccggttgacccagtctccatccttcctgtctgcatctgtaggagacagagtcaccatcacttgtcgtgccagtcagggcattaggaattattttgcctggtatcagcaaaaaccagggaaagctcctaacctcctgatctatgctgcatccttgttgcaaagtggggtcccatcaaggttcagcggcagtggatttgggacagatttcactctcaccatcagcagcctgcagcctgaagactttgcaacttactattgtcaacaggctagcagtttcccttttactttcggtggagggaccaaggtggagatcaaaaggaccgtggctgcccccagcgtgttcatcttccctcctagcgacgagcagctgaagagcggcaccgctagcgtggtgtgtctgctgaataacttctatcccagggaggccaaggtgcagtggaaggtggataacgccctgcagagcggcaactcccaggagtccgtgaccgagcaggactccaaggacagcacctactccctgagctccaccctgaccctgtccaaggctgattatgagaagcacaaggtgtatgcttgcgaggtgacacaccagggcctgtccagccctgtgaccaagagcttcaaccggggcgagtgc

SEQ ID NO: 22 - DNA, 642 bp, Synthetic construct
  source 1..642: mol_type = other DNA
gccatccggttgacccagtctccatccttcctgtctgcatctgtaggagacagagtcaccatcacttgtcgtgccagtcagggcattaggaattatttagcctggtatcagcaaaaaccagggaaagctcctaacctcctgatctatgctgcatccttgttgcaaagtggggtcccatcaaggttcagcggcagtggatttgggacagatttcactctcaccatcagcagcctgcagcctgaagactttgcaacttactattgtcaagatgctagcagtttccctctcactttcggtggagggaccaaggtggagatcaaaaggaccgtggctgcccccagcgtgttcatcttccctcctagcgacgagcagctgaagagcggcaccgctagcgtggtgtgtctgctgaataacttctatcccagggaggccaaggtgcagtggaaggtggataacgccctgcagagcggcaactcccaggagtccgtgaccgagcaggactccaaggacagcacctactccctgagctccaccctgaccctgtccaaggctgattatgagaagcacaaggtgtatgcttgcgaggtgacacaccagggcctgtccagccctgtgaccaagagcttcaaccggggcgagtgc

SEQ ID NO: 23 - DNA, 642 bp, Synthetic construct
  source 1..642: mol_type = other DNA
gccatccggttgacccagtctccatccttcctgtctgcatctgtaggagacagagtcaccatcacttgtcgtgccagtcagggcattaggaattatttagcctggtatcagcaaaaaccagggaaagctcctaacctcctgatctatgctgcatccttgttgcaaagtggggtcccatcaaggttcagcggcagtggatttgggacagatttcactctcaccatcagcagcctgcagcctgaagactttgcaacttactattgtcaacaggctagcagtttccctgttcatttcggtggagggaccaaggtggagatcaaaaggaccgtggctgcccccagcgtgttcatcttccctcctagcgacgagcagctgaagagcggcaccgctagcgtggtgtgtctgctgaataacttctatcccagggaggccaaggtgcagtggaaggtggataacgccctgcagagcggcaactcccaggagtccgtgaccgagcaggactccaaggacagcacctactccctgagctccaccctgaccctgtccaaggctgattatgagaagcacaaggtgtatgcttgcgaggtgacacaccagggcctgtccagccctgtgaccaagagcttcaaccggggcgagtgc

SEQ ID NO: 24 - DNA, 366 bp, Synthetic construct
  source 1..366: mol_type = other DNA
gaggtgcagctgttggagtctgggggaggcgtggtccagcctgggaggtccctgagactctcctgtgcagcctctggattcatgtttagtaactattggatgatttgggtccgtcaggctgcagggaagggtcctgagtgggtggcagttatatcatatgatggaagcaataaattgtacgcagactccgtgaagggccgattcaccatctccagagacaattccaagaacacgctgtatctgcaaatgaacagcctgagagctgaggacactgccgtgtattactgtgcgagagtaccagtaggttactactactactacggtatggacgtctggggtcaggggactccggtcaccgtctcatca

SEQ ID NO: 25 - DNA, 366 bp, Synthetic construct
  source 1..366: mol_type = other DNA
gaggtgcagctgttggagtctgggggaggcgtggtccagcctgggaggtccctgagactctcctgtgcagcctctggattcatgtttagtaactattggatgatttgggtccgtcaggctgcagggaagggtcctgagtgggtggcagttatatcatatgatggaagcaataaattgtacgcagactccgtgaagggccgattcaccatctccagagacaattccaagaacacgctgtatctgcaaatgaacagcctgagagctgaggacactgccgtgtattactgtgcgagagtaccagtaggttactactactactactcgatggacgtctggggtcaggggactccggtcaccgtctcatca

SEQ ID NO: 26 - DNA, 366 bp, Synthetic construct
  source 1..366: mol_type = other DNA
gaggtgcagctgttggagtctgggggaggcgtggtccagcctgggaggtccctgagactctcctgtgcagcctctggattcatgtttagtaactattggatgatttgggtccgtcaggctgcagggaagggtcctgagtgggtggcagttatatcatatgatggaagcaataaattgtacgcagactccgtgaagggccgattcaccatctccagagacaattccaagaacacgctgtatctgcaaatgaacagcctgagagctgaggacactgccgtgtattactgtgcgagagtaccaattgcttactactactactacggtatggacgtctggggtcaggggactccggtcaccgtctcatca

SEQ ID NO: 27 - DNA, 321 bp, Synthetic construct
  source 1..321: mol_type = other DNA
gccatccggttgacccagtctccatccttcctgtctgcatctgtaggagacagagtcaccatcacttgtcgtgccagtcagggcattaggaattatttagcctggtatcagcaaaaaccagggaaagctcctaacctcctgatctatgctgcatccttgttgcaaagtggggtcccatcaaggttcagcggcagtggatttgggacagatttcactctcaccatcagcagcctgcagcctgaagactttgcaacttactattgtcaacaggctagcagtttccctctcactttcggtggagggaccaaggtggagatcaaa

SEQ ID NO: 28 - DNA, 321 bp, Synthetic construct
  source 1..321: mol_type = other DNA
gccatccggttgacccagtctccatccttcctgtctgcatctgtaggagacagagtcaccatcacttgtcgtgccagtcagggcattaggaattattttgcctggtatcagcaaaaaccagggaaagctcctaacctcctgatctatgctgcatccttgttgcaaagtggggtcccatcaaggttcagcggcagtggatttgggacagatttcactctcaccatcagcagcctgcagcctgaagactttgcaacttactattgtcaacaggctagcagtttcccttttactttcggtggagggaccaaggtggagatcaaa

SEQ ID NO: 29 - DNA, 321 bp, Synthetic construct
  source 1..321: mol_type = other DNA
gccatccggttgacccagtctccatccttcctgtctgcatctgtaggagacagagtcaccatcacttgtcgtgccagtcagggcattaggaattatttagcctggtatcagcaaaaaccagggaaagctcctaacctcctgatctatgctgcatccttgttgcaaagtggggtcccatcaaggttcagcggcagtggatttgggacagatttcactctcaccatcagcagcctgcagcctgaagactttgcaacttactattgtcaagatgctagcagtttccctctcactttcggtggagggaccaaggtggagatcaaa

SEQ ID NO: 30 - DNA, 321 bp, Synthetic construct
  source 1..321: mol_type = other DNA
gccatccggttgacccagtctccatccttcctgtctgcatctgtaggagacagagtcaccatcacttgtcgtgccagtcagggcattaggaattatttagcctggtatcagcaaaaaccagggaaagctcctaacctcctgatctatgctgcatccttgttgcaaagtggggtcccatcaaggttcagcggcagtggatttgggacagatttcactctcaccatcagcagcctgcagcctgaagactttgcaacttactattgtcaacaggctagcagtttccctgttcatttcggtggagggaccaaggtggagatcaaa

SEQ ID NO: 31 - protein, 452 aa, Synthetic construct
EVQLLESGGGVVQPGRSLRLSCAASGFMFSNYWMIWVRQAAGKGPEWVAVISYDGSNKLYADSVKGRFTISRDNSKNTLYLQMNSLRAEDTAVYYCARVPVGYYYYYGMDVWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 452 aa, Synthetic construct
EVQLLESGGGVVQPGRSLRLSCAASGFMFSNYWMIWVRQAAGKGPEWVAVISYDGSNKLYADSVKGRFTISRDNSKNTLYLQMNSLRAEDTAVYYCARVPVGYYYYYSMDVWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 452 aa, Synthetic construct
EVQLLESGGGVVQPGRSLRLSCAASGFMFSNYWMIWVRQAAGKGPEWVAVISYDGSNKLYADSVKGRFTISRDNSKNTLYLQMNSLRAEDTAVYYCARVPIAYYYYYGMDVWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 214 aa, Synthetic construct
AIRLTQSPSFLSASVGDRVTITCRASQGIRNYLAWYQQKPGKAPNLLIYAASLLQSGVPSRFSGSGFGTDFTLTISSLQPEDFATYYCQQASSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 214 aa, Synthetic construct
AIRLTQSPSFLSASVGDRVTITCRASQGIRNYFAWYQQKPGKAPNLLIYAASLLQSGVPSRFSGSGFGTDFTLTISSLQPEDFATYYCQQASSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 214 aa, Synthetic construct
AIRLTQSPSFLSASVGDRVTITCRASQGIRNYLAWYQQKPGKAPNLLIYAASLLQSGVPSRFSGSGFGTDFTLTISSLQPEDFATYYCQDASSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 214 aa, Synthetic construct
AIRLTQSPSFLSASVGDRVTITCRASQGIRNYLAWYQQKPGKAPNLLIYAASLLQSGVPSRFSGSGFGTDFTLTISSLQPEDFATYYCQQASSFPVHFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 122 aa, Synthetic construct
EVQLLESGGGVVQPGRSLRLSCAASGFMFSNYWMIWVRQAAGKGPEWVAVISYDGSNKLYADSVKGRFTISRDNSKNTLYLQMNSLRAEDTAVYYCARVPVGYYYYYGMDVWGQGTPVTVSS

SEQ ID NO: 39 - protein, 122 aa, Synthetic construct
EVQLLESGGGVVQPGRSLRLSCAASGFMFSNYWMIWVRQAAGKGPEWVAVISYDGSNKLYADSVKGRFTISRDNSKNTLYLQMNSLRAEDTAVYYCARVPVGYYYYYSMDVWGQGTPVTVSS

SEQ ID NO: 40 - protein, 122 aa, Synthetic construct
EVQLLESGGGVVQPGRSLRLSCAASGFMFSNYWMIWVRQAAGKGPEWVAVISYDGSNKLYADSVKGRFTISRDNSKNTLYLQMNSLRAEDTAVYYCARVPIAYYYYYGMDVWGQGTPVTVSS

SEQ ID NO: 41 - protein, 107 aa, Synthetic construct
AIRLTQSPSFLSASVGDRVTITCRASQGIRNYLAWYQQKPGKAPNLLIYAASLLQSGVPSRFSGSGFGTDFTLTISSLQPEDFATYYCQQASSFPLTFGGGTKVEIK

SEQ ID NO: 42 - protein, 107 aa, Synthetic construct
AIRLTQSPSFLSASVGDRVTITCRASQGIRNYFAWYQQKPGKAPNLLIYAASLLQSGVPSRFSGSGFGTDFTLTISSLQPEDFATYYCQQASSFPFTFGGGTKVEIK

SEQ ID NO: 43 - protein, 107 aa, Synthetic construct
AIRLTQSPSFLSASVGDRVTITCRASQGIRNYLAWYQQKPGKAPNLLIYAASLLQSGVPSRFSGSGFGTDFTLTISSLQPEDFATYYCQDASSFPLTFGGGTKVEIK

SEQ ID NO: 44 - protein, 107 aa, Synthetic construct
AIRLTQSPSFLSASVGDRVTITCRASQGIRNYLAWYQQKPGKAPNLLIYAASLLQSGVPSRFSGSGFGTDFTLTISSLQPEDFATYYCQQASSFPVHFGGGTKVEIK